(19) United States
(12) Patent Application Publication
(10) Pub. No.: US 2024/0059760 A1
(43) Pub. Date: Feb. 22, 2024

(54) POLYPEPTIDES FOR DETECTION AND TREATMENT OF SARS-COV-2

(71) Applicant: The University of Chicago, Chicago, IL (US)

(72) Inventors: Anthony KOSSIAKOFF, Chicago, IL (US); Tomasz SLEZAK, Chicago, IL (US)

(73) Assignee: The University of Chicago, Chicago, IL (US)

(21) Appl. No.: 18/251,351

(22) PCT Filed: Nov. 1, 2021
(86) PCT No.: PCT/US2021/072152

§ 371 Date: May 1, 2023

Related U.S. Application Data
(60) Provisional application No. 63/108,614, filed on Nov. 2, 2020.

(51) Int. Cl. C07K 16/10; A61P 31/14; G01N 33/569

(52) U.S. Cl. CPC C07K 16/1003; A61P 31/14; G01N 33/56983; C07K 2317/21; C07K 2317/24; C07K 2317/626; G01N 2333/165

(57) ABSTRACT

Aspects of the present disclosure relate to polypeptides that specifically bind to a SARS-CoV-2 spike protein, and to methods of use for treatment, prevention, and diagnosis of a SARS-CoV-2 infection. Certain aspects are directed to antibodies and fragments thereof, including Fabs, configured to bind to various epitopes within the receptor binding domain (RBD) of a SARS-CoV-2 spike protein. Compositions, kits, and methods for detection of SARS-CoV-2 are also disclosed.

### CROSS-REFERENCE TO RELATED APPLICATIONS

This application claims the benefit of priority to U.S. Provisional Patent Application No. 63/108,614, filed Nov. 2, 2020, incorporated herein by reference in entirety.

### SEQUENCE LISTING

The instant application contains a Sequence Listing which has been submitted in ASCII format and is hereby incorporated by reference in its entirety. Said ASCII copy, created on Oct. 10, 2021, is named ARCD_P0712WO_Sequence_Listing.txt and is 168,217 bytes in size.

### BACKGROUND

### I. Field of the Invention

Aspects of the invention relate to at least the fields of virology and molecular biology.

### II. Background

SARS-CoV-2, the virus that causes COVID-19, is highly infectious and can cause severe and life-threatening disease. SARS-CoV-2 infection is mediated by the receptor binding domain (RBD) of the SARS-CoV-2 spike protein binding to the ACE2 molecule on the surface of a cell. Molecules capable of inhibiting such an interaction may be useful in treatment, prevention, and/or diagnosis of COVID-19.

There is a need in the art for compositions and methods for treatment and prevention of SARS-CoV-2 infection. Also recognized is a need for methods for detection of SARS-CoV-2.

### SUMMARY

Aspects of the present disclosure are directed to polypeptides (e.g., antibodies, antibody fragments, Fabs, etc.) capable of binding to to a SARS-CoV-2 spike protein, polypeptides that specifically bind to a SARS-CoV-2 spike protein, compositions comprising such polypeptides, and methods of use for treatment, prevention, and diagnosis of a SARS-CoV-2 infection.

Embodiments of the disclosure include polypeptides, antibodies, antibody fragments, Fabs, SARS-CoV-2 spike protein-binding proteins, anti-SARS-CoV-2 spike protein antibodies, anti-SARS-CoV-2 spike protein Fabs, methods for treatment of a SARS-CoV-2 infection, methods for prevention of a SARS-CoV-2 infection, methods for reducing severity of a SARS-CoV-2 infection, methods for diagnosis of a SARS-CoV-2 infection, methods for inhibiting entry of a SARS-CoV-2 virus into a cell, methods for inhibiting an interaction between a SARS-CoV-2 spike protein and an ACE2 protein, nucleic acids, vectors, cells, pharmaceutical compositions, and kits. Because the SARS-COV-2 virus causes COVID-19, any embodiment discussed in the context of SARS-COV-2 can be implemented with respect to COVID-19.

Compositions of the disclosure can include at least 1, 2, 3, 4, 5, or more of the following components: polypeptides, antibodies, antibody fragments, Fabs, cells, cell populations, therapeutic agents, anti-viral agents, excipients, nucleic acids, vectors, enzymes, fluorophores, and substrates. One or more of these components may be specifically excluded from certain embodiments.

Methods of the disclosure can include at least 1, 2, 3, 4, 5 or more of the following steps: administering a polypeptide to a subject, administering an antibody to a subject, administering a Fab to a subject, administering an anti-viral agent to a subject, administering a cell to a subject, diagnosing a subject as having a SARS-CoV-2 infection, obtaining a sample from a subject, detecting a SARS-CoV-2 virus in a sample, culturing a cell, transfecting a cell, introducing a nucleic acid into a cell, detecting a detection pair, generating a SARS-CoV-2 spike protein-binding polypeptide, and generating an anti-SARS-CoV-2 antibody or antigen binding fragment thereof. One or more of these steps may be specifically excluded from certain embodiments.

Disclosed herein, in some embodiments, is a polypeptide that specifically binds to a SARS-CoV-2 spike protein, comprising: (a) a CDR-L1 comprising a sequence having at least 80% identity to SEQ ID NO:15; (ii) a CDR-L2 comprising a sequence having at least 80% identity to SEQ ID NO:16; and (iii) a CDR-L3 comprising a sequence having at least 80% identity to SEQ ID NO:17, SEQ ID NO:18, SEQ ID NO:19, SEQ ID NO:20, SEQ ID NO:21, SEQ ID NO:22, or SEQ ID NO:23. In some embodiments, the CDR-H1 comprises a sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.5, 99.9, or 100% identity to SEQ ID NO:1, SEQ ID NO:2, SEQ ID NO:3, SEQ ID NO:4, or SEQ ID NO:5. In some embodiments, the CDR-H2 comprises a sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.5, 99.9, or 100% identity to SEQ ID NO:6, SEQ ID NO:113, or SEQ ID NO:7. In some embodiments, the CDR-H3 comprises a sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.5, 99.9, or 100% identity to SEQ ID NO:8, SEQ ID NO:9, SEQ ID NO:10, SEQ ID NO:11, SEQ ID NO:12, SEQ ID NO:13, or SEQ ID NO:14. In some embodiments, the CDR-L1 comprises a sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.5, 99.9, or 100% identity to SEQ ID NO:15. In some embodiments, the CDR-L2 comprises a sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.5, 99.9, or 100% identity to SEQ ID NO:16. In some embodiments, the CDR-L3 comprises a sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.5, 99.9, or 100% identity to SEQ ID NO:17, SEQ ID NO:18, SEQ ID NO:19, SEQ ID NO:20, SEQ ID NO:21, SEQ ID NO:22, or SEQ ID NO:23.

In some embodiments, the CDR-H1 comprises SEQ ID NO:1, SEQ ID NO:2, SEQ ID NO:3, SEQ ID NO:4, or SEQ ID NO:5. In some embodiments, the CDR-H1 comprises SEQ ID NO:1. In some embodiments, the CDR-H1 comprises SEQ ID NO:2. In some embodiments, the CDR-H1 comprises SEQ ID NO:3. In some embodiments, the CDR-H1 comprises SEQ ID NO:4. In some embodiments, the CDR-H1 comprises SEQ ID NO:5.

In some embodiments, the CDR-H2 comprises SEQ ID NO:6, SEQ ID NO:113, or SEQ ID NO:7. In some embodiments, the CDR-H2 comprises SEQ ID NO:6. In some embodiments, the CDR-H2 comprises SEQ ID NO:113. In some embodiments, the CDR-H2 comprises SEQ ID NO:7.

In some embodiments, the CDR-H3 comprises SEQ ID NO:8, SEQ ID NO:9, SEQ ID NO:10, SEQ ID NO:11, SEQ ID NO:12, SEQ ID NO:13, or SEQ ID NO:14. In some embodiments, the CDR-H3 comprises SEQ ID NO:8. In some embodiments, the CDR-H3 comprises SEQ ID NO:9. In some embodiments, the CDR-H3 comprises SEQ ID NO:10. In some embodiments, the CDR-H3 comprises SEQ ID NO:11. In some embodiments, the CDR-H3 comprises SEQ ID NO:12. In some embodiments, the CDR-H3 comprises SEQ ID NO:13. In some embodiments, the CDR-H3 comprises SEQ ID NO:14.

In some embodiments, the CDR-L1 comprises SEQ ID NO:15. In some embodiments, the CDR-L2 comprises SEQ ID NO:16. In some embodiments, the CDR-L3 comprises SEQ ID NO:17, SEQ ID NO:18, SEQ ID NO:19, SEQ ID NO:20, SEQ ID NO:21, SEQ ID NO:22, or SEQ ID NO:23. In some embodiments, the CDR-L3 comprises SEQ ID NO:17. In some embodiments, the CDR-L3 comprises SEQ ID NO:18. In some embodiments, the CDR-L3 comprises SEQ ID NO:19. In some embodiments, the CDR-L3 comprises SEQ ID NO:20. In some embodiments, the CDR-L3 comprises SEQ ID NO:21. In some embodiments, the CDR-L3 comprises SEQ ID NO:22. In some embodiments, the CDR-L3 comprises SEQ ID NO:23.

In some embodiments, the VH comprises a sequence having at least 80% identity to SEQ ID NO:44, SEQ ID NO:46, SEQ ID NO:48, SEQ ID NO:50, SEQ ID NO:52, SEQ ID NO:54, or SEQ ID NO:56. In some embodiments, the VH comprises a sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.5, 99.9, or 100% identity to SEQ ID NO:44, SEQ ID NO:46, SEQ ID NO:48, SEQ ID NO:50, SEQ ID NO:52, SEQ ID NO:54, or SEQ ID NO:56. In some embodiments, the VH comprises SEQ ID NO:44, SEQ ID NO:46, SEQ ID NO:48, SEQ ID NO:50, SEQ ID NO:52, SEQ ID NO:54, or SEQ ID NO:56. In some embodiments, the VH comprises SEQ ID NO:44. In some embodiments, the VH comprises SEQ ID NO:46. In some embodiments, the VH comprises SEQ ID NO:48. In some embodiments, the VH comprises SEQ ID NO:50. In some embodiments, the VH comprises SEQ ID NO:52. In some embodiments, the VH comprises SEQ ID NO:54. In some embodiments, the VH comprises SEQ ID NO:56.

In some embodiments, the VL comprises a sequence having at least 80% identity to SEQ ID NO:45, SEQ ID NO:47, SEQ ID NO:49, SEQ ID NO:51, SEQ ID NO:53, SEQ ID NO:55, or SEQ ID NO:57. In some embodiments, the VL comprises a sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.5, 99.9, or 100% identity to SEQ ID NO:45, SEQ ID NO:47, SEQ ID NO:49, SEQ ID NO:51, SEQ ID NO:53, SEQ ID NO:55, or SEQ ID NO:57. In some embodiments, the VL comprises SEQ ID NO:45, SEQ ID NO:47, SEQ ID NO:49, SEQ ID NO:51, SEQ ID NO:53, SEQ ID NO:55, or SEQ ID NO:57. In some embodiments, the VL comprises SEQ ID NO:45. In some embodiments, the VL comprises SEQ ID NO:47. In some embodiments, the VL comprises SEQ ID NO:49. In some embodiments, the VL comprises SEQ ID NO:51. In some embodiments, the VL comprises SEQ ID NO:53. In some embodiments, the VL comprises SEQ ID NO:55. In some embodiments, the VL comprises SEQ ID NO:57.

Disclosed herein, in some embodiments, is a polypeptide that specifically binds to a SARS-CoV-2 spike protein, comprising a CDR-H1 comprising SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, or SEQ ID NO:43. In some embodiments, the CDR-H1 comprises a sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.5, 99.9, or 100% identity to SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, or SEQ ID NO:43. In some embodiments, the CDR-H1 comprises SEQ ID NO:24. In some embodiments, the CDR-H1 comprises SEQ ID NO:25. In some embodiments, the CDR-H1 comprises SEQ ID NO:26. In some embodiments, the CDR-H1 comprises SEQ ID NO:27. In some embodiments, the CDR-H1 comprises SEQ ID NO:28. In some embodiments, the CDR-H1 comprises SEQ ID NO:29. In some embodiments, the CDR-H1 comprises SEQ ID NO:30. In some embodiments, the CDR-H1 comprises SEQ ID NO:31. In some embodiments, the CDR-H1 comprises SEQ ID NO:32. In some embodiments, the CDR-H1 comprises SEQ ID NO:33. In some embodiments, the CDR-H1 comprises SEQ ID NO:34. In some embodiments, the CDR-H1 comprises SEQ ID NO:35. In some embodiments, the CDR-H1 comprises SEQ ID NO:36. In some embodiments, the CDR-H1 comprises SEQ ID NO:37. In some embodiments, the CDR-H1 comprises SEQ ID NO:38. In some embodiments, the polypeptide has an affinity for the SARS-CoV-2 spike protein of at most 110 pM. In some embodiments, the CDR-H1 comprises SEQ ID NO:39. In some embodiments, the CDR-H1 comprises SEQ ID NO:40. In some embodiments, the CDR-H1 comprises SEQ ID NO:41. In some embodiments, the CDR-H1 comprises SEQ ID NO:42. In some embodiments, the polypeptide has an affinity for the SARS-CoV-2 spike protein of at most 350 pM. In some embodiments, the CDR-H1 comprises SEQ ID NO:43. In some embodiments, the polypeptide further comprises a CDR-H2 comprising SEQ ID NO:6, a CDR-H3 comprising SEQ ID NO:8, a CDR-L1 comprising SEQ ID NO:15, a CDR-L2 comprising SEQ ID NO:16, and a CDR-L3 comprising SEQ ID NO:17.

In some embodiments, the polypeptide has an affinity for a SARS-CoV-2 spike protein of at most, at least, or about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 20.5, 21, 21.5, 22, 22.5, 23, 23.5, 24, 24.5, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, or 1000 nM, or any range derivable therein.

In some embodiments, the polypeptide comprises a VL comprising a sequence having at least 80% identity to SEQ ID NO:45. In some embodiments, the VL comprises a sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.5, 99.9, or 100% identity to SEQ ID NO:45. In some embodiments, the VL comprises SEQ ID NO:45.

Disclosed herein, in some embodiments, is a polypeptide that specifically binds to a SARS-CoV-2 spike protein, comprising (a) a sequence having at least 80% identity to one of SEQ ID NOs:77-83; and (b) a sequence having at least 80% identity to one of SEQ ID NOs:84-109. In some embodiments, the polypeptide comprises a sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.5, 99.9, or 100% identity to one of SEQ ID NOs:77-83. In some embodiments, the polypeptide comprises one of SEQ ID NOs:77-83. In some embodiments, the polypeptide comprises a sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.5, 99.9, or 100% identity to one of SEQ ID NOs:84-109. In some embodiments, the polypeptide comprises one of SEQ ID NOs: 84-109.

In some embodiments, the polypeptide specifically binds to a receptor-binding domain (RBD) of the SARS-CoV-2 spike protein. In some embodiments, the polypeptide is capable of inhibiting entry of a SARS-CoV-2 virus into a cell. In some embodiments, the polypeptide is not capable of inhibiting entry of a SARS-CoV-2 virus into a cell. In some embodiments, the polypeptide is an antibody or antigen-binding fragment thereof. In some embodiments, the polypeptide is a human antibody, humanized antibody, recombinant antibody, chimeric antibody, an antibody derivative, a veneered antibody, a diabody, a monoclonal antibody, or a polyclonal antibody. In some embodiments, the antibody is a monoclonal antibody. In some embodiments, the antibody is a human antibody. In some embodiments, the polypeptide is a Fab.

Also disclosed are nucleic acids encoding for a polypeptide disclosed herein and vectors comprising such nucleic acids. Further embodiments are directed to a cell or a population of cells comprising polypeptides, nucleic acids, and/or vectors of the present disclosure. Pharmaceutical compositions are also described comprising polypeptides, nucleic acids, vectors, and/or cells of the disclosure and a pharmaceutically acceptable excipient. In some embodiments, the pharmaceutical composition further comprises an additional therapeutic. In some embodiments, the additional therapeutic is an additional anti-viral agent.

Disclosed herein, in some embodiments, is a method of generating a polypeptide of the disclosure comprising culturing a cell comprising a nucleic acid encoding for the polypeptide and subjecting the cell to conditions sufficient to express the polypeptide from the nucleic acid. Conditions sufficient to express a polypeptide from a nucleic acid in a cell are known in the art, certain examples of which are described elsewhere herein.

Disclosed herein, in some embodiments, is a method for treating a subject for a SARS-CoV-2 infection comprising administering to a subject an effective amount of a polypeptide, as disclosed herein, that specifically binds to a SARS-CoV-2 spike protein. In some embodiments, the polypeptide comprises (a) one of SEQ ID NOs:77-83 and (b) one of SEQ ID NOs: 84-109. In some embodiments, the polypeptide comprises SEQ ID NO:77 and SEQ ID NO:84. In some embodiments, the polypeptide comprises SEQ ID NO:77 and SEQ ID NO:105.

Also disclosed herein, in some embodiments, is a method for detecting a SARS-CoV-2 spike protein in a sample, the method comprising (a) providing to the sample (i) a first polypeptide operatively linked to a first component of a detection pair; and (ii) a second polypeptide operatively linked to a second component of the detection pair; and (b) detecting the detection pair, wherein the first polypeptide specifically binds to a first region of the SARS-CoV-2 spike protein and the second polypeptide specifically binds to a second region of the SARS-CoV-2 spike protein. In some embodiments, the detection pair comprises an enzyme and detecting the detection pair comprises detecting enzymatic activity of the enzyme. In some embodiments, the detection pair comprises a TEM-1 β-lactamase. In some embodiments, the first component of the detection pair comprises a BLF1 fragment of the TEM-1 β-lactamase. In some embodiments, the second component of the detection pair comprises a BLF2 fragment of the TEM-1 β-lactamase. In some embodiments, the detection pair comprises a donor fluorophore and an acceptor fluorophore. In some embodiments, detecting the detection pair comprises detecting fluorescence from the acceptor fluorophore. In some embodiments, the first polypeptide is operatively linked to the first component of the detection pair via protein G or a variant thereof. In some embodiments, the second polypeptide is operatively linked to the second component of the detection pair via protein G or a variant thereof. In some embodiments, the first polypeptide comprises SEQ ID NO:77 and SEQ ID NO:84. In some embodiments, the first polypeptide comprises SEQ ID NO:77 and SEQ ID NO:105. In some embodiments, the second polypeptide comprises SEQ ID NO:83 and SEQ ID NO:90.

Also disclosed herein, in some embodiments, is a kit comprising (a) a first polypeptide operatively linked to a first component of a detection pair; and (b) a second polypeptide operatively linked to a second component of the detection pair, wherein the first polypeptide specifically binds to a first region of a SARS-CoV-2 spike protein and the second polypeptide specifically binds to a second region of the SARS-CoV-2 spike protein. In some embodiments, the kit further comprises instructions for detecting the SARS-CoV-2 spike protein in a sample. In some embodiments, the detection pair comprises an enzyme and detecting the detection pair comprises detecting enzymatic activity of the enzyme. In some embodiments, the detection pair comprises a TEM-1 β-lactamase. In some embodiments, the first component of the detection pair comprises a BLF1 fragment of the TEM-1 β-lactamase. In some embodiments, the second component of the detection pair comprises a BLF2 fragment of the TEM-1 β-lactamase. In some embodiments, the kit further comprises a substrate for the enzyme.

Because the SARS-CoV-2 virus causes COVID-19, any embodiment discussed in the context of SARS-CoV-2 can be implemented with respect to COVID-19.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

“Individual, “subject,” and “patient” are used interchangeably and can refer to a human or non-human.

It is specifically contemplated that any limitation discussed with respect to one embodiment of” the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows results from single-point phage ELISA of RBD binders selected by phage display. Clones with strong signal against RBD (termed sRBD1-sRBD7) were chosen for further sequencing and characterization. Data points show average value from 3 independent experiments. Error bars display standard deviation (SD) from the mean.

FIGS. 2A and 2B show characterization of Fabs of the present disclosure against RBD. FIG. 2A shows a SPR sensogram showing the fast on-fast off kinetics between sRBD1 and RBD. The concentration of Fab was serially diluted two-fold for each run starting at 200 nM. FIG. 2B shows a sensogram showing slower association and slower dissociation binding between sRBD7 and RBD. The concentration of Fab was serially diluted two-fold for each run starting at 200 nM.

FIG. 3A shows a schematic of a neutralization mechanism model. Anti-RBD synthetic antibodies (or fragments thereof) block the interaction with human ACE2 receptor on the cell surface. FIG. 3B shows a schematic of a neutralization model with pG mediated avidity enhancement. FIG. 3C shows the protection from cell death achieved by different concentrations of sRBD1 and sRBD7 Fabs. Neutralization IC₅₀ value for sRBD1 is 4.8 nM. No efficient neutralization with sRBD7 was observed. Results were normalized. Experiment was done in triplicate and the error bars display SD value from the mean.

FIGS. 4A-4C demonstrate affinity maturation of sRBD1. FIG. 4A shows a WebLogo plot of the affinity matured CDR-H1 variants. Original sequence is presented at the top. FIG. 4B shows a sRBD1.15 SPR sensogram showing the improved slow-dissociation rate.

The concentration of Fab was serially diluted two-fold for each run starting at 25 nM. FIG. 4C shows the improvement in SARS CoV-2 neutralization. IC₅₀ of sRBD1.15 was 10-times improved relative to sRBD1 with a value of 0.48 nM. Results were normalized. Experiment was done in triplicate and the error bars display SD value from the mean.

FIGS. 5A and 5B show a GA1-FAB^{LRT} protein complementation assay. FIG. 5A shows a model of SARS-CoV-2 detection using GA1-FAB^{LRT} protein complementation assay. Two separate fragments of BL enzyme are attached to two different Fabs via protein GA1-LRT interaction. BL fragments can associate to form an active enzyme state when Fabs bind to the Receptor Binding Domain (RBD). FIG. 5B shows detection of SARS-CoV-2 at different concentrations using complementary parts: GA1-BLF1/sRBD7 and BLF2-GA1/sRBD1. Detectable signal was observed from 15 to 250 nM. At higher concentrations the hook effect is observed where each Fab recognizes separate RBD molecule, leading to signal decrease. Reaction was incubated for 20 minutes at room temperature. Results were normalized. The experiment was done in triplicate and the error bars display SD value from the mean.

FIG. 6 shows epitope binning by SPR.

FIG. 7 shows a SPR single injection of sRBD1 on immobilized His-6 SARS-CoV-2 S protein. The injection was done with 100 nM sRBD1 Fab. Dissociation rate on immobilized Spike protein is slower than on immobilized RBD due to possible avidity effect generated by trimeric conformation of S protein.

FIG. 8A shows results from GA1-FAB^{LRT} protein complementation assay with affinity matured sRBD1.5 Fab. Detection of SARS-CoV-2 at different concentrations using complementary parts: pGA1-BLF1/sRBD7 and BLF2-pGA1/sRBD1.5. Detectable signal was observed from 7 to 125 nM. At higher concentration the hook effect is observed. Reaction was incubated for 20 minutes at RT; results were normalized. Experiment was done in triplicate and the error bars represent SD value from the mean. FIG. 8B shows results from GA1-FAB^{LRT} protein complementation assay controls. No signal was detected when the same sRBD fab was premixed with both BLF constructs (construct 1 and 2). 250 nM RBD was used in the assay and the measurement was taken after 20 minutes at room temperature and the results were normalized. Constructs: (1)-sRBD1/BLF1+sRBD1/BLF2; (2)-sRBD7/BLF1+sRBD7/BLF2; (3)-sRBD1/BLF2+sRBD7/BLF1.

FIG. 9 show results from a GA1-FAB^{LRT} protein complementation assay on a full-length SARS-CoV-2 Spike protein. Detection of SARS-CoV-2 at different concentrations using complementary parts: GA1-BLF1/sRBD7 and BLF2-GA1/sRBD1. Detectable signal was observed from 7 to 125 nM. At higher concentrations the hook effect was observed when each Fab recognized separate RBD molecules, leading to signal decrease. Reaction was incubated for 20 minutes at room temperature. Results were normalized.

FIG. 10 shows that virus neutralization improved over 2.5-times by sRBD1 dimerization via protein G-dimer. IC₅₀ neutralization for sRBD1 and dimerized sRBD1 were 4.8 nM and 1.9 nM respectively. Results were normalized. Experiment was done in triplicate and the error bars display SD value from the mean.

FIG. 11 shows results from GA1-FAB^{LRT} protein complementation assay controls for Spike detection. No signal was detected when the same RBD Fab was premixed with both BLF constructs (BLF1 and BLF2). 10 nM Spike was used in the assay and the measurement was taken after 20 minutes at RT and the results were normalized. Experiment was done in triplicate and the error bars represent SD value from the mean. Constructs: (1)-sRBD1/BLF1+sRBD1/BLF2; (2)-sRBD7/BLF1+sRBD7/BLF2; (3)-sRBD1/BLF2+sRBD7/BLF1.

FIGS. 12A and 12B demonstrate post lyophilization stability of Fabs. FIG. 12A shows results from single-point ELISA of Fabs after reconstitution from lyophilization. No difference in binding was observed. Data points show average value from 3 independent experiments. Error bars represent SD value from the mean. FIG. 12B shows results from GA1-LRT protein complementation assay after reconstitution from lyophilization. No difference was observed when lyophilization was done with BSA. 40% signal reduction was observed when samples were freeze-dried in PBS, but in the absence of BSA. Reaction was incubated for 20 minutes at RT; results were normalized. Data points show average value from 3 independent experiments. Error bars represent SD value from the mean.

FIGS. 13A and 13B show single-point ELISA with ACE2 competition. RBD was immobilized on high binding plate and the Fabs' binding was evaluated with and without 100 nM ACE2 competition. FIG. 13A shows a schematic of an ELISA competition model. FIG. 13B shows results from the single-point ELISA. Results show the competition for a binding site between RBD1 and ACE2. Level of ACE2 competition decrease for affinity matured variants sRBD1.3 and sRBD1.5. Data points show average value from 3 independent experiments. Error bars represent SD value from the mean.

### DETAILED DESCRIPTION OF THE INVENTION

The present disclosure fulfils certain needs in the art by providing compositions and methods for treatment, prevention, diagnosis, and detection of SARS-CoV-2. Aspects of the disclosure are based on the generation of novel polypeptides that specifically bind to a SARS-CoV-2 spike protein. Certain polypeptides of the disclosure bind to the receptor binding domain (RBD) of a SARS-CoV-2 spike protein with high affinity and are capable of inhibiting entry of SARS-CoV-2 into a cell. Such polypeptides are useful, for example, in methods for treatment and prevention of a SARS-CoV-2 infection. Certain polypeptides of the disclosure bind to the RBD of a SARS-CoV-2 spike protein and are not capable of inhibiting entry of SARS-CoV-2 into a cell. Such polypeptides are useful, for example, in methods for detection of a SARS-CoV-2 virus in a sample (e.g., a biological sample from a subject).

### I. Antibodies

Aspects of the disclosure relate to antibodies, or fragments thereof (e.g., Fabs), capable of specifically binding to a SARS-CoV-2 spike (S) protein. Certain aspects relate to antibodies, or fragments thereof, that specifically bind to a receptor binding domain (RBD) of a SARS-CoV-2 spike protein.

The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (k). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (0, delta (6), gamma (γ), alpha (a), or epsilon (c) chains, respectively. IgG has several subtypes, including, but not limited to, IgG1, IgG2, IgG3, and IgG4. IgM subtypes include IgM1 and IgM2. IgA subtypes include IgA1 and IgA2.

A. Types of Antibodies

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bispecific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-L1, CDR-L2, and CDR-L3. The L3 (CDR-L3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as CDR-H1, CDR-H2 and CDR-H3. The majority of amino acid residues in the variable domains, or Fv fragments (VH and VL), are part of the framework regions. The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include:

1) Computational predictions of the tertiary structure of the antibody/epitope binding interactions based on the chemical nature of the amino acid sequence of the antibody variable region and composition of the epitope.

2) Hydrogen-deuterium exchange and mass spectroscopy

3) Polypeptide fragmentation and peptide mapping approaches in which one generates multiple overlapping peptide fragments from the full length of the polypeptide and evaluates the binding affinity of these peptides for the epitope.

4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity.

Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

B. Functional Antibody Fragments and Antigen-Binding Fragments

1. Antigen-Binding Fragments

Certain aspects relate to antibody fragments, such as antibody fragments that bind to a SARS-CoV-2 spike protein. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, N Y (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015), each of which are incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment (also “Fab”) means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

2. Fragment Antigen Binding Region, Fab

Fab polypeptides of the disclosure include the Fab antigen binding fragment of an antibody. Unless specifically stated otherwise, the term “Fab” relates to a polypeptide excluding the Fc portion of the antibody. The Fab may be conjugated to a polypeptide comprising other components, such as further antigen binding domains, costimulatory domains, linkers, peptide spacers, transmembrane domains, endodomains, and accessory proteins. Fab polypeptides can be generated using conventional techniques known in the art and are well-described in the literature.

3. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

C. Polypeptides with antibody CDRs & Scaffolding Domains that Display the CDRs

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

D. Antibody Binding

The term “selective binding agent” refers to a molecule that binds to an antigen. Non-limiting examples include antibodies, antigen-binding fragments, scFv, Fab, Fab′, F(ab′)2, single chain antibodies, peptides, peptide fragments and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

1. Affinity/Avidity

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. Affinity can be expressed as, for example, 20-fold greater binding ability of the antibody to its antigen then to an unrelated amino acid sequence. As used herein, the term “avidity” refers to the resistance of a complex of two or more agents to dissociation after dilution. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (KD or Kd), using the equation KD=koff/kon=[A][B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the KD are mol/L (molarity, or M), or concentration. The Ka of an antibody is the opposite of the KD, and is determined by the equation Ka=1/KD. Examples of some experimental methods that can be used to determine the KD value are: enzyme-linked immunosorbent assays (ELISA), isothermal titration calorimetry (ITC), fluorescence anisotropy, surface plasmon resonance (SPR), and affinity capillary electrophoresis (ACE). The affinity constant (Ka) of an antibody is the opposite of the KD, and is determined by the equation Ka=1/KD.

Antibodies deemed useful in certain embodiments may have an affinity constant (Ka) of about, at least about, or at most about 10⁶, 10⁷, 10⁸, 10⁹, or 10¹⁰ M or any range derivable therein. Similarly, in some embodiments, antibodies may have a dissociation constant of about, at least about or at most about 10⁻⁶, 10⁻⁷, 10⁻⁸, 10⁻⁹, 10⁻¹⁰ M, or any range derivable therein. These values are reported for antibodies discussed herein and the same assay may be used to evaluate the binding properties of such antibodies. An antibody of the invention is said to “specifically bind” its target antigen when the dissociation constant (KD) is ≤10⁻⁸ M. The antibody specifically binds antigen with “high affinity” when the KD is ≤5×10^{31 9} M, and with “very high affinity” when the KD is ≤5×10⁻¹⁰ M.

2. Epitope Specificity

The epitope of an antigen is the specific region of the antigen for which an antibody has binding affinity. In the case of protein or polypeptide antigens, the epitope is the specific residues (or specified amino acids or protein segment) that the antibody binds with high affinity. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B and/or T cells respond or recognize. Polypeptide epitopes can be formed from both contiguous amino acids and noncontiguous amino acids juxtaposed by tertiary folding of a polypeptide. An epitope typically includes at least 3, and typically 5-10 amino acids in a unique spatial conformation.

Epitope specificity of an antibody can be determined in a variety of ways. One approach, for example, involves testing a collection of overlapping peptides of 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N and C terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

3. Modification of Antibody Antigen-Binding Domains

It is understood that the antibodies of the present invention may be modified, such that they are substantially identical to the antibody polypeptide sequences, or fragments thereof, and still bind the epitopes of the present invention. Polypeptide sequences are “substantially identical” when optimally aligned using such programs as Clustal Omega, IGBLAST, GAP or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present invention, providing that the variations in the amino acid sequence maintain at least 75%, more preferably at least 80%, at least 90%, at least 95%, at least 96%, at least 97%, at least 98% and most preferably at least 99% sequence identity. In particular, conservative amino acid replacements are contemplated.

Conservative replacements are those that take place within a family of amino acids that are related in their side chains. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). For example, it is reasonable to expect that an isolated replacement of a leucine moiety with an isoleucine or valine moiety, or a similar replacement of an amino acid with a structurally related amino acid in the same family, will not have a major effect on the binding or properties of the resulting molecule, especially if the replacement does not involve an amino acid within a framework site. Whether an amino acid change results in a functional peptide can readily be determined by assaying the specific activity of the polypeptide derivative. Standard ELISA, Surface Plasmon Resonance (SPR), or other antibody binding assays can be performed by one skilled in the art to make a quantitative comparison of antigen binging affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art; Dill and McCallum., Science 338:1042-1046 (2012). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the invention.

Framework modifications can be made to antibodies to decrease immunogenicity, for example, by “backmutating” one or more framework residues to a corresponding germline sequence.

It is also contemplated that the antigen-binding domain may be multi-specific or multivalent by multimerizing the antigen-binding domain with VH and VL region pairs that bind either the same antigen (multi-valent) or a different antigen (multi-specific).

E. Chemical Modification of Antibodies

In some aspects, also contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861). In certain embodiments, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other embodiments, one or more new N-linked glycosylation sites are created. Antibodies typically have an N-linked glycosylation site in the Fc region.

Additional antibody variants include cysteine variants, wherein one or more cysteine residues in the parent or native amino acid sequence are deleted from or substituted with another amino acid (e.g., serine). Cysteine variants are useful, inter alia, when antibodies must be refolded into a biologically active conformation. Cysteine variants may have fewer cysteine residues than the native antibody and typically have an even number to minimize interactions resulting from unpaired cysteines.

In some aspects, the polypeptides can be pegylated to increase biological half-life by reacting the polypeptide with polyethylene glycol (PEG) or a reactive ester or aldehyde derivative of PEG, under conditions in which one or more PEG groups become attached to the polypeptide. Polypeptide pegylation may be carried out by an acylation reaction or an alkylation reaction with a reactive PEG molecule (or an analogous reactive water-soluble polymer). Methods for pegylating proteins are known in the art and can be applied to the polypeptides of the invention to obtain PEGylated derivatives of antibodies. See, e.g., EP 0 154 316 and EP 0 401 384. In some aspects, the antibody is conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, propropylene glycol homopolymers, polypropylene oxide/ethylene oxide co-polymers, polyoxyethylated polyols, and polyvinyl alcohols. As used herein, the term “polyethylene glycol” is intended to encompass any of the forms of PEG that have been used to derivatize other proteins.

1. Conjugation

Derivatives of the antibodies and antigen binding fragments that are described herein are also provided. The derivatized antibody or fragment thereof may comprise any molecule or substance that imparts a desired property to the antibody or fragment. The derivatized antibody can comprise, for example, a detectable (or labeling) moiety (e.g., a radioactive, colorimetric, antigenic, or enzymatic molecule, or a detectable bead), a molecule that binds to another molecule (e.g., biotin or streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses).

Optionally, an antibody or an immunological portion of an antibody can be chemically conjugated to, or expressed as, a fusion protein with other proteins. In some aspects, polypeptides may be chemically modified by conjugating or fusing the polypeptide to serum protein, such as human serum albumin, to increase half-life of the resulting molecule. See, e.g., EP 0322094 and EP 0 486 525. In some aspects, the polypeptides may be conjugated to a diagnostic agent and used diagnostically, for example, to monitor the development or progression of a disease and determine the efficacy of a given treatment regimen. In some aspects, the polypeptides may also be conjugated to a therapeutic agent to provide a therapy in combination with the therapeutic effect of the polypeptide. Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. The functional nucleic acid molecules may act as effectors, inhibitors, modulators, and stimulators of a specific activity possessed by a target molecule, or the functional nucleic acid molecules may possess a de novo activity independent of any other molecules.

In some aspects, disclosed are antibodies and antibody-like molecules that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules include toxins, therapeutic enzymes, antibiotics, radiolabeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands.

a. Conjugate Types

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, FITC, rhodamine, dansyl, umbelliferone, dimethyl acridinium ester (DMAE), Texas red, luminol, NADPH and α- or β-galactosidase. Antibody conjugates include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference. Molecules containing azido groups may also be used to form covalent bonds to proteins through reactive nitrene intermediates that are generated by low intensity ultraviolet light (Potter & Haley, 1983).

In some aspects, contemplated are immunoconjugates comprising an antibody or antigen-binding fragment thereof conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In this way, the agent of interest can be targeted directly to cells bearing cell surface antigen. The antibody and agent may be associated through non-covalent interactions such as through electrostatic forces, or by covalent bonds. Various linkers, known in the art, can be employed in order to form the immunoconjugate. Additionally, the immunoconjugate can be provided in the form of a fusion protein. In one aspect, an antibody may be conjugated to various therapeutic substances in order to target the cell surface antigen. Examples of conjugated agents include, but are not limited to, metal chelate complexes, drugs, toxins and other effector molecules, such as cytokines, lymphokines, chemokines, immunomodulators, radiosensitizers, asparaginase, carboranes, and radioactive halogens.

In antibody drug conjugates (ADC), an antibody (Ab) is conjugated to one or more drug moieties (D) through a linker (L). The ADC may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. Antibody drug conjugates may also be produced by modification of the antibody to introduce electrophilic moieties, which can react with nucleophilic substituents on the linker reagent or drug. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate. In yet another aspect, the antibody may be conjugated to a “receptor” (such as streptavidin) for utilization in tumor or cancer cell pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

Examples of an antibody-drug conjugates known to a person skilled in the art are pro-drugs useful for the local delivery of cytotoxic or cytostatic agents, i.e. drugs to kill or inhibit tumor cells in the treatment of cancer (Syrigos and Epenetos, Anticancer Res. 19:605-614 (1999); Niculescu-Duvaz and Springer, Adv. Drg. Del. Rev. 26:151-172 (1997); U.S. Pat. No. 4,975,278). In contrast, systematic administration of these unconjugated drug agents may result in unacceptable levels of toxicity to normal cells as well as the target tumor cells (Baldwin et al., Lancet 1:603-5 (1986); Thorpe, (1985) “Antibody Carriers of Cytotoxic Agents in Cancer Therapy: A Review,” In: Monoclonal Antibodies '84: Biological and Clinical Applications, A. Pincera et al., (eds.) pp. 475-506). Both polyclonal antibodies and monoclonal antibodies have been reported as useful in these strategies (Rowland et al., Cancer Immunol. Immunother. 21:183-87 (1986)).

In certain aspects, ADC include covalent or aggregative conjugates of antibodies, or antigen-binding fragments thereof, with other proteins or polypeptides, such as by expression of recombinant fusion proteins comprising heterologous polypeptides fused to the N-terminus or C-terminus of an antibody polypeptide. For example, the conjugated peptide may be a heterologous signal (or leader) polypeptide, e.g., the yeast alpha-factor leader, or a peptide such as an epitope tag (e.g., V5-His). Antibody-containing fusion proteins may comprise peptides added to facilitate purification or identification of the antibody (e.g., poly-His). An antibody polypeptide also can be linked to the FLAG® (Sigma-Aldrich, St. Louis, Mo.) peptide as described in Hopp et al., Bio/Technology 6:1204 (1988), and U.S. Pat. No. 5,011,912. Oligomers that contain one or more antibody polypeptides may be employed as antagonists. Oligomers may be in the form of covalently linked or non-covalently linked dimers, trimers, or higher oligomers. Oligomers comprising two or more antibody polypeptides are contemplated for use. Other oligomers include heterodimers, homotrimers, heterotrimers, homotetramers, heterotetramers, etc. In certain aspects, oligomers comprise multiple antibody polypeptides joined via covalent or non-covalent interactions between peptide moieties fused to the antibody polypeptides. Such peptides may be peptide linkers (spacers), or peptides that have the property of promoting oligomerization. Leucine zippers and certain polypeptides derived from antibodies are among the peptides that can promote oligomerization of antibody polypeptides attached thereto, as described in more detail below.

b. Conjugation Methodology

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates may also be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bos(p-diazoniumbenzoyl)-ethylenediamine), diisocyanates (such as toluene 2,6-diisocyanate), and bis-active fluorine compounds (such as 1,5-difluoro-2,4-dinitrobenzene). In some aspects, derivatization of immunoglobulins by selectively introducing sulfhydryl groups in the Fc region of an immunoglobulin, using reaction conditions that do not alter the antibody combining site, are contemplated. Antibody conjugates produced according to this methodology are disclosed to exhibit improved longevity, specificity, and sensitivity (U.S. Pat. No. 5,196,066, incorporated herein by reference). Site-specific attachment of effector or reporter molecules, wherein the reporter or effector molecule is conjugated to a carbohydrate residue in the Fc region has also been disclosed in the literature (O'Shannessy et al., 1987).

F. Proteins

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID 1-121.

In some embodiments, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOs:1-109 or 113-121.

In some embodiments, the polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of SEQ ID NOs:1-109 or 113-121.

In some embodiments, the polypeptide may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOs:1-109 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS:1-109 or 113-121.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOS:1-121 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOS:1-121.

In some embodiments, a polypeptide (e.g., antibody, antibody fragment, Fab, etc.) of the disclosure comprises a CDR that is at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical (or any range derivable therein) in sequence to one of SEQ ID NOS:1-43 or 113. In some embodiments, a polypeptide comprises 1, 2, and/or 3 CDRs from one of SEQ ID NOS:44-76. The CDR may be one that has been determined by Kabat, IMGT, or Chothia. In further embodiments, a polypeptide may have CDRs that have 1, 2, and/or 3 amino acid changes (e.g., addition of 1 or 2 amino acids, deletions of 1 or 2 amino acids, substitution) with respect to these 1, 2, or 3 CDRs. In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

From amino to carboxy terminus the CDRs are CDR1, CDR2, and CDR3. In some embodiments, a polypeptide may have CDRs that have 1, 2, and/or 3 amino acid changes (e.g., addition of 1 or 2 amino acids, deletions of 1 or 2 amino acids, substitution) with respect to CDR1, CDR2, or CDR3. In some embodiments, the CDRs of SEQ ID NOS:44-76 may further comprise 1, 2, 3, 4, 5, or 6 additional amino acids at the amino or carboxy terminus of the CDR, The additional amino acids may be from the heavy and/or light chain framework regions of SEQ ID NOS:44-76, that are shown as immediately adjacent to the CDRs. Accordingly, embodiments relate to polypeptides comprising an HCDR1 (i.e., CDR-H1), HCDR2 (i.e., CDR-H2), HCDR3 (i.e., CDR-H3), LCDR1 (i.e., CDR-L1), LCDR2 (i.e., CDR-L2), and/or LCDR3 (i.e., CDR-L3) with at least or at most or exactly 1, 2, 3, 4, 5, 6 or 7 amino acids at the amino end of the CDR or at the carboxy end of the CDR, wherein the additional amino acids are the 1, 2, 3, 4, 5, 6, or 7 amino acids of SEQ ID NOS:44-76 that are shown as immediately adjacent to the CDRs. Other embodiments relate to antibodies comprising one or more CDRs, wherein the CDR is a fragment of SEQ ID NO:1-43 and wherein the fragment lacks 1, 2, 3, 4, or 5 amino acids from the amino or carboxy end of the CDR. In some embodiments, the CDR may lack one, 2, 3, 4, 5, 6, or 7 amino acids from the carboxy end and may further comprise 1, 2, 3, 4, 5, 6, 7, or 8 amino acids from the framework region of the amino end of the CDR. In some embodiments, the CDR may lack one, 2, 3, 4, 5, 6, or 7 amino acids from the amino end and may further comprise 1, 2, 3, 4, 5, 6, 7, or 8 amino acids from the framework region of the carboxy end of the CDR. In further embodiments, an antibody may be alternatively or additionally humanized in regions outside the CDR(s) and/or variable region(s). In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

In other embodiments, a polypeptide or protein comprises 1, 2, 3, 4, 5, or 6 CDRs from either or both of the light and heavy variable regions of SEQ ID NOS:44-76, and 1, 2, 3, 4, 5, or 6 CDRs may have 1, 2, and/or 3 amino acid changes with respect to these CDRs. In some embodiments, parts or all of the antibody sequence outside the variable region have been humanized. A protein may comprise one or more polypeptides. In some aspects, a protein may contain one or two polypeptides similar to a heavy chain polypeptide and/or 1 or 2 polypeptides similar to a light chain polypeptide.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

1. Sequences

Sequences from certain polypeptides, including SARS-CoV-2 binding polypeptides, disclosed herein are provided in SEQ ID NOs: 1-109 and in Tables 1 and 2 below.

TABLE 1

| | SEQ | |
|---|---|---|
| | ID | |
| Polypeptide | NO: | Sequence |
| SRBD1 | 1 | FSSSYI |
| CDR-H1 | | |
| SRBD2 | 2 | VYSSYI |
| CDR-H1 | | |
| sRBD3 | 3 | IYSSYI |
| CDR-H1 | | |
| SRBD4 | 4 | LYSSYI |
| CDR-H1 | | |
| SRBD5 | 2 | VYSSYI |
| CDR-H1 | | |
| SRBD6 | 5 | VSSSYI |
| CDR-H1 | | |
| SRBD7 | 2 | VYSSYI |
| CDR-H1 | | |
| SRBD1 | 6 | SISSYSGYTY |
| CDR-H2 | | |
| SRBD2 | 113 | YISSYYGSTY |
| CDR-H2 | | |
| SRBD3 | 113 | YISSYYGSTY |
| CDR-H2 | | |
| SRBD4 | 113 | YISSYYGSTY |
| CDR-H2 | | |
| SRBD5 | 113 | YISSYYGSTY |
| CDR-H2 | | |
| SRBD6 | 113 | YISSYYGSTY |
| CDR-H2 | | |
| SRBD7 | 7 | YISPYYGSTY |
| CDR-H2 | | |
| SRBD1 | 8 | SYQGQEAL |
| CDR-H3 | | |
| SRBD2 | 9 | YAGHAM |
| CDR-H3 | | |
| SRBD3 | 10 | YGDDAM |
| CDR-H3 | | |
| SRBD4 | 11 | YHSWAF |
| CDR-H3 | | |
| SRBD5 | 12 | YSYPGGM |
| CDR-H3 | | |
| SRBD6 | 13 | YAEGMGM |
| CDR-H3 | | |
| SRBD7 | 14 | YWSYGGM |
| CDR-H3 | | |
| SRBD1- | 15 | SVSSA |
| SRBD7 | | |
| CDR-L1 | | |
| SRBD1- | 16 | SASSLYS |
| SRBD7 | | |
| CDR-L2 | | |
| SRBD1 | 17 | YLYFSLV |
| CDR-L3 | | |
| SRBD2 | 18 | SNYSYPI |
| CDR-L3 | | |
| SRBD3 | 19 | ASWGSPI |
| CDR-L3 | | |
| SRBD4 | 20 | SYYGSLI |
| CDR-L3 | | |
| SRBD5 | 21 | SYYGNLI |
| CDR-L3 | | |
| SRBD6 | 22 | HAWGYLI |
| CDR-L3 | | |
| sRBD7 | 23 | YYYGALI |
| CDR-L3 | | |
| SRBD1.1 | 24 | FGGSYI |
| CDR-H1 | | |
| SRBD1.2 | 25 | FHGIYL |
| CDR-H1 | | |
| SRBD1.3 | 26 | FHSTYI |
| CDR-H1 | | |
| SRBD1.4 | 27 | FWLGYI |
| CDR-H1 | | |
| SRBD1.5 | 28 | FWNSYI |
| CDR-H1 | | |
| SRBD1.6 | 29 | FWTAYI |
| CDR-H1 | | |
| SRBD1.7 | 30 | FFNSYI |
| CDR-H1 | | |
| SRBD1.8 | 31 | FHNIYL |
| CDR-H1 | | |
| SRBD1.9 | 32 | FHNLYV |
| CDR-H1 | | |
| SRBD1.10 | 33 | FHRAYI |
| CDR-H1 | | |
| SRBD1.11 | 34 | FHRIYI |
| CDR-H1 | | |
| SRBD1.12 | 35 | FWASYI |
| CDR-H1 | | |
| SRBD1.13 | 36 | FYASYI |
| CDR-H1 | | |
| SRBD1.14 | 37 | LANNYV |
| CDR-H1 | | |
| SRBD1.15 | 38 | FHNSYI |
| CDR-H1 | | |
| SRBD1.16 | 39 | FHRTYI |
| CDR-H1 | | |
| SRBD1.17 | 40 | LNNIYI |
| CDR-H1 | | |
| SRBD1.18 | 41 | VHWVYF |
| CDR-H1 | | |
| SRBD1.19 | 42 | LHNQYL |
| CDR-H1 | | |
| SRBD1.1- | 43 | F/L/VH/W/A/F/G/N/YN/R/A/G/L/S/T/ |
| 1.19 | | WS/I/A/T/G/L/N/Q/VYI/L/V/F |
| CDR-H1 | | |
| consensus | | |
| SRBD1 VH | 44 | EISEVQLVESGGGLVQPGGSLRLSCAASGFNFSSSYIHWVRQAP |
| | | GKGLEWVASISSYSGYTYYADSVKGRFTISADTSKNTAYLQMN |
| | | SLRAEDTAVYYCARSYQGQEALDYWGQGTLVTVSS |
| SRBD1 VL | 45 | SDIQMTQSPSSLSASVGDRVTITCRASQSVSSAVAWYQQKPGK |
| | | APKLLIYSASSLYSGVPSRFSGSRSGTDFTLTISSLQPEDFATYYC |
| | | QQYLYFSLVTFGQGTKVEIK |
| sRBD2 VH | 46 | EISEVQLVESGGGLVQPGGSLRLSCAASGENVYSSYIHWVRQAP |
| | | GKGLEWVAYISSYYGSTYYADSVKGRFTISADTSKNTAYLQMN |
| | | SLRAEDTAVYYCARYAGHAMDYWGQGTLVTVSS |
| SRBD2 VL | 47 | SDIQMTQSPSSLSASVGDRVTITCRASQSVSSAVAWYQQKPGK |
| | | APKLLIYSASSLYSGVPSRFSGSRSGTDFTLTISSLQPEDFATYYC |
| | | QQSNYSYPITFGQGTKVEIK |
| SRBD3 VH | 48 | EISEVQLVESGGGLVQPGGSLRLSCAASGFNIYSSYIHWVRQAP |
| | | GKGLEWVAYISSYYGSTYYADSVKGRFTISADTSKNTAYLQMN |
| | | SLRAEDTAVYYCARYGDDAMDYWGQGTLVTVSS |
| SRBD3 VL | 49 | SDIQMTQSPSSLSASVGDRVTITCRASQSVSSAVAWYQQKPGK |
| | | APKLLIYSASSLYSGVPSRFSGSRSGTDFTLTISSLQPEDFATYYC |
| | | QQASWGSPITFGQGTKVEIK |
| sRBD4 VH | 50 | EISEVQLVESGGGLVQPGGSLRLSCAASGFNLYSSYIHWVRQAP |
| | | GKGLEWVAYISSYYGSTYYADSVKGRFTISADTSKNTAYLQMN |
| | | SLRAEDTAVYYCARYHSWAFDYWGQGTLVTVSS |
| SRBD4 VL | 51 | SDIQMTQSPSSLSASVGDRVTITCRASQSVSSAVAWYQQKPGK |
| | | APKLLIYSASSLYSGVPSRFSGSRSGTDFTLTISSLQPEDFATYYC |
| | | QQSYYGSLITFGQGTKVEIK |
| SRBD5 VH | 52 | EISEVQLVESGGGLVQPGGSLRLSCAASGFNVYSSYIHWVRQAP |
| | | GKGLEWVAYISSYYGSTYYADSVKGRFTISADTSKNTAYLQMN |
| | | SLRAEDTAVYYCARYSYPGGMDYWGQGTLVTVSS |
| SRBD5 VL | 53 | SDIQMTQSPSSLSASVGDRVTITCRASQSVSSAVAWYQQKPGK |
| | | APKLLIYSASSLYSGVPSRFSGSRSGTDFTLTISSLQPEDFATYYC |
| | | QQSYYGNLITFGQGTKVEIK |
| SRBD6 VH | 54 | EISEVQLVESGGGLVQPGGSLRLSCAASGFNVSSSYIHWVRQAP |
| | | GKGLEWVAYISSYYGSTYYADSVKGRFTISADTSKNTAYLQMN |
| | | SLRAEDTAVYYCARYAEGMGMDYWGQGTLVTVSS |
| SRBD6 VL | 55 | SDIQMTQSPSSLSASVGDRVTITCRASQSVSSAVAWYQQKPGK |
| | | APKLLIYSASSLYSGVPSRFSGSRSGTDFTLTISSLQPEDFATYYC |
| | | QQHAWGYLITFGQGTKVEIK |
| SRBD7 VH | 56 | EISEVQLVESGGGLVQPGGSLRLSCAASGFNVYSSYIHWVRQAP |
| | | GKGLEWVAYISPYYGSTYYADSVKGRFTISADTSKNTAYLQMN |
| | | SLRAEDTAVYYCARYWSYGGMDYWGQGTLVTVSS |
| SRBD7 VL | 57 | SDIQMTQSPSSLSASVGDRVTITCRASQSVSSAVAWYQQKPGK |
| | | APKLLIYSASSLYSGVPSRFSGSRSGTDFTLTISSLQPEDFATYYC |
| | | QQYYYGALITFGQGTKVEIK |
| SRBD1.1 | 58 | EISEVQLVESGGGLVQPGGSLRLSCAASGFNFGGSYIHWVRQAP |
| VH | | GKGLEWVASISSYSGYTYYADSVKGRFTISADTSKNTAYLQMN |
| | | SLRAEDTAVYYCARSYQGQEALDYWGQGTLVTVSS |
| SRBD1.1 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1.2 | 59 | EISEVQLVESGGGLVQPGGSLRLSCAASGFNFHGIYLHWVRQAP |
| VH | | GKGLEWVASISSYSGYTYYADSVKGRFTISADTSKNTAYLQMN |
| | | SLRAEDTAVYYCARSYQGQEALDYWGQGTLVTVSS |
| SRBD1.2 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1.3 | 60 | EISEVQLVESGGGLVQPGGSLRLSCAASGFNFHSTYIHWVRQAP |
| VH | | GKGLEWVASISSYSGYTYYADSVKGRFTISADTSKNTAYLQMN |
| | | SLRAEDTAVYYCARSYQGQEALDYWGQGTLVTVSS |
| SRBD1.3 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1.4 | 61 | EISEVQLVESGGGLVQPGGSLRLSCAASGFNFWLGYIHWVRQA |
| VH | | PGKGLEWVASISSYSGYTYYADSVKGRFTISADTSKNTAYLQM |
| | | NSLRAEDTAVYYCARSYQGQEALDYWGQGTLVTVSS |
| SRBD1.4 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1.5 | 62 | EISEVQLVESGGGLVQPGGSLRLSCAASGFNFWNSYIHWVRQA |
| VH | | PGKGLEWVASISSYSGYTYYADSVKGRFTISADTSKNTAYLQM |
| | | NSLRAEDTAVYYCARSYQGQEALDYWGQGTLVTVSS |
| SRBD1.5 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1.6 | 63 | EISEVQLVESGGGLVQPGGSLRLSCAASGFNFWTAYIHWVRQA |
| VH | | PGKGLEWVASISSYSGYTYYADSVKGRFTISADTSKNTAYLQM |
| | | NSLRAEDTAVYYCARSYQGQEALDYWGQGTLVTVSS |
| SRBD1.6 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1.7 | 64 | EISEVQLVESGGGLVQPGGSLRLSCAASGFNFFNSYIHWVRQAP |
| VH | | GKGLEWVASISSYSGYTYYADSVKGRFTISADTSKNTAYLQMN |
| | | SLRAEDTAVYYCARSYQGQEALDYWGQGTLVTVSS |
| SRBD1.7 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1.8 | 65 | EISEVQLVESGGGLVQPGGSLRLSCAASGFNFHNIYLHWVRQAP |
| VH | | GKGLEWVASISSYSGYTYYADSVKGRFTISADTSKNTAYLQMN |
| | | SLRAEDTAVYYCARSYQGQEALDYWGQGTLVTVSS |
| SRBD1.8 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1.9 | 66 | EISEVQLVESGGGLVQPGGSLRLSCAASGFNFHNLYVHWVRQA |
| VH | | PGKGLEWVASISSYSGYTYYADSVKGRFTISADTSKNTAYLQM |
| | | NSLRAEDTAVYYCARSYQGQEALDYWGQGTLVTVSS |
| SRBD1.9 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1.10 | 67 | EISEVQLVESGGGLVQPGGSLRLSCAASGFNFHRAYIHWVRQAP |
| VH | | GKGLEWVASISSYSGYTYYADSVKGRFTISADTSKNTAYLQMN |
| | | SLRAEDTAVYYCARSYQGQEALDYWGQGTLVTVSS |
| SRBD1.10 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1.11 | 68 | EISEVQLVESGGGLVQPGGSLRLSCAASGFNFHRIYIHWVRQAP |
| VH | | GKGLEWVASISSYSGYTYYADSVKGRFTISADTSKNTAYLQMN |
| | | SLRAEDTAVYYCARSYQGQEALDYWGQGTLVTVSS |
| SRBD1.11 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1.12 | 69 | EISEVQLVESGGGLVQPGGSLRLSCAASGFNFWASYIHWVRQA |
| VH | | PGKGLEWVASISSYSGYTYYADSVKGRFTISADTSKNTAYLQM |
| | | NSLRAEDTAVYYCARSYQGQEALDYWGQGTLVTVSS |
| SRBD1.12 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1.13 | 70 | EISEVQLVESGGGLVQPGGSLRLSCAASGFNFYASYIHWVRQAP |
| VH | | GKGLEWVASISSYSGYTYYADSVKGRFTISADTSKNTAYLQMN |
| | | SLRAEDTAVYYCARSYQGQEALDYWGQGTLVTVSS |
| SRBD1.13 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1.14 | 71 | EISEVQLVESGGGLVQPGGSLRLSCAASGFNLANNYVHWVRQA |
| VH | | PGKGLEWVASISSYSGYTYYADSVKGRFTISADTSKNTAYLQM |
| | | NSLRAEDTAVYYCARSYQGQEALDYWGQGTLVTVSS |
| SRBD1.14 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1.15 | 72 | EISEVQLVESGGGLVQPGGSLRLSCAASGFNFHNSYIHWVRQAP |
| VH | | GKGLEWVASISSYSGYTYYADSVKGRFTISADTSKNTAYLQMN |
| | | SLRAEDTAVYYCARSYQGQEALDYWGQGTLVTVSS |
| SRBD1.15 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1.16 | 73 | EISEVQLVESGGGLVQPGGSLRLSCAASGFNFHRTYIHWVRQAP |
| VH | | GKGLEWVASISSYSGYTYYADSVKGRFTISADTSKNTAYLQMN |
| | | SLRAEDTAVYYCARSYQGQEALDYWGQGTLVTVSS |
| SRBD1.16 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1.17 | 74 | EISEVQLVESGGGLVQPGGSLRLSCAASGFNLNNIYIHWVRQAP |
| VH | | GKGLEWVASISSYSGYTYYADSVKGRFTISADTSKNTAYLQMN |
| | | SLRAEDTAVYYCARSYQGQEALDYWGQGTLVTVSS |
| SRBD1.17 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1.18 | 75 | EISEVQLVESGGGLVQPGGSLRLSCAASGFNVHWVYFHWVRQ |
| VH | | APGKGLEWVASISSYSGYTYYADSVKGRFTISADTSKNTAYLQ |
| | | MNSLRAEDTAVYYCARSYQGQEALDYWGQGTLVTVSS |
| SRBD1.18 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1.19 | 76 | EISEVQLVESGGGLVQPGGSLRLSCAASGFNLHNQYLHWVRQA |
| VH | | PGKGLEWVASISSYSGYTYYADSVKGRFTISADTSKNTAYLQM |
| | | NSLRAEDTAVYYCARSYQGQEALDYWGQGTLVTVSS |
| SRBD1.19 | 45 | SDIQMTQSPSSLSASVGDRVTITCRASQSVSSAVAWYQQKPGK |
| VL | | APKLLIYSASSLYSGVPSRFSGSRSGTDFTLTISSLQPEDFATYYC |
| | | QQYLYFSLVTFGQGTKVEIK |
| SRBD1 Full | 84 | EISEVQLVESGGGLVQPGGSLRLSCAASGFNFSSSYIHWVRQAP |
| heavy chain | | GKGLEWVASISSYSGYTYYADSVKGRFTISADTSKNTAYLQMN |
| | | SLRAEDTAVYYCARSYQGQEALDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD1 Full | 77 | SDIQMTQSPSSLSASVGDRVTITCRASQSVSSAVAWYQQKPGK |
| light chain | | APKLLIYSASSLYSGVPSRFSGSRSGTDFTLTISSLQPEDFATYYC |
| | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD2 Full | 85 | EISEVQLVESGGGLVQPGGSLRLSCAASGENVYSSYIHWVRQAP |
| heavy chain | | GKGLEWVAYISSYYGSTYYADSVKGRFTISADTSKNTAYLQMN |
| | | SLRAEDTAVYYCARYAGHAMDYWGQGTLVTVSSASTKGPSVF |
| | | PLAPSSKSTSGGTAALGCLVKDYFPEPVTVSWNSGALTSGVHTF |
| | | PAVLQSSGLYSLSSVVTVPSSSLGTQTYICNVNHKPSNTKVDKK |
| | | VEPKSCDKTHT |
| SRBD2 Full | 78 | SDIQMTQSPSSLSASVGDRVTITCRASQSVSSAVAWYQQKPGK |
| light chain | | APKLLIYSASSLYSGVPSRFSGSRSGTDFTLTISSLQPEDFATYYC |
| | | QQSNYSYPITFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD3 Full | 86 | EISEVQLVESGGGLVQPGGSLRLSCAASGFNIYSSYIHWVRQAP |
| heavy chain | | GKGLEWVAYISSYYGSTYYADSVKGRFTISADTSKNTAYLQMN |
| | | SLRAEDTAVYYCARYGDDAMDYWGQGTLVTVSSASTKGPSVF |
| | | PLAPSSKSTSGGTAALGCLVKDYFPEPVTVSWNSGALTSGVHTF |
| | | PAVLQSSGLYSLSSVVTVPSSSLGTQTYICNVNHKPSNTKVDKK |
| | | VEPKSCDKTHT |
| SRBD3 Full | 79 | SDIQMTQSPSSLSASVGDRVTITCRASQSVSSAVAWYQQKPGK |
| light chain | | APKLLIYSASSLYSGVPSRFSGSRSGTDFTLTISSLQPEDFATYYC |
| | | QQASWGSPITFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| sRBD4 Full | 87 | EISEVQLVESGGGLVQPGGSLRLSCAASGFNLYSSYIHWVRQAP |
| heavy chain | | GKGLEWVAYISSYYGSTYYADSVKGRFTISADTSKNTAYLQMN |
| | | SLRAEDTAVYYCARYHSWAFDYWGQGTLVTVSSASTKGPSVF |
| | | PLAPSSKSTSGGTAALGCLVKDYFPEPVTVSWNSGALTSGVHTF |
| | | PAVLQSSGLYSLSSVVTVPSSSLGTQTYICNVNHKPSNTKVDKK |
| | | VEPKSCDKTHT |
| SRBD4 Full | 80 | SDIQMTQSPSSLSASVGDRVTITCRASQSVSSAVAWYQQKPGK |
| light chain | | APKLLIYSASSLYSGVPSRFSGSRSGTDFTLTISSLQPEDFATYYC |
| | | QQSYYGSLITFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD5 Full | 88 | EISEVQLVESGGGLVQPGGSLRLSCAASGENVYSSYIHWVRQAP |
| heavy chain | | GKGLEWVAYISSYYGSTYYADSVKGRFTISADTSKNTAYLQMN |
| | | SLRAEDTAVYYCARYSYPGGMDYWGQGTLVTVSSASTKGPSV |
| | | FPLAPSSKSTSGGTAALGCLVKDYFPEPVTVSWNSGALTSGVHT |
| | | FPAVLQSSGLYSLSSVVTVPSSSLGTQTYICNVNHKPSNTKVDK |
| | | KVEPKSCDKTHT |
| SRBD5 Full | 81 | SDIQMTQSPSSLSASVGDRVTITCRASQSVSSAVAWYQQKPGK |
| light chain | | APKLLIYSASSLYSGVPSRFSGSRSGTDFTLTISSLQPEDFATYYC |
| | | QQSYYGNLITFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD6 Full | 89 | EISEVQLVESGGGLVQPGGSLRLSCAASGFNVSSSYIHWVRQAP |
| heavy chain | | GKGLEWVAYISSYYGSTYYADSVKGRFTISADTSKNTAYLQMN |
| | | SLRAEDTAVYYCARYAEGMGMDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| sRBD6 Full | 82 | SDIQMTQSPSSLSASVGDRVTITCRASQSVSSAVAWYQQKPGK |
| light chain | | APKLLIYSASSLYSGVPSRFSGSRSGTDFTLTISSLQPEDFATYYC |
| | | QQHAWGYLITFGQGTKVEIKRTVAAPSVFIFPPSDSQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |
| SRBD7 Full | 90 | EISEVQLVESGGGLVQPGGSLRLSCAASGENVYSSYIHWVRQAP |
| heavy chain | | GKGLEWVAYISPYYGSTYYADSVKGRFTISADTSKNTAYLQMN |
| | | SLRAEDTAVYYCARYWSYGGMDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD7 Full | 83 | SDIQMTQSPSSLSASVGDRVTITCRASQSVSSAVAWYQQKPGK |
| light chain | | APKLLIYSASSLYSGVPSRFSGSRSGTDFTLTISSLQPEDFATYYC |
| | | QQYYYGALITFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.1 | 91 | EISEVQLVESGGGLVQPGGSLRLSCAASGFNFGGSYIHWVRQAP |
| Full heavy | | GKGLEWVASISSYSGYTYYADSVKGRFTISADTSKNTAYLQMN |
| chain | | SLRAEDTAVYYCARSYQGQEALDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD1.1 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.2 | 92 | EISEVQLVESGGGLVQPGGSLRLSCAASGFNFHGIYLHWVRQAP |
| Full heavy | | GKGLEWVASISSYSGYTYYADSVKGRFTISADTSKNTAYLQMN |
| chain | | SLRAEDTAVYYCARSYQGQEALDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD1.2 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.3 | 93 | EISEVQLVESGGGLVQPGGSLRLSCAASGFNFHSTYIHWVRQAP |
| Full heavy | | GKGLEWVASISSYSGYTYYADSVKGRFTISADTSKNTAYLQMN |
| chain | | SLRAEDTAVYYCARSYQGQEALDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD1.3 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.4 | 94 | EISEVQLVESGGGLVQPGGSLRLSCAASGFNFWLGYIHWVRQA |
| Full heavy | | PGKGLEWVASISSYSGYTYYADSVKGRFTISADTSKNTAYLQM |
| chain | | NSLRAEDTAVYYCARSYQGQEALDYWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD1.4 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.5 | 95 | EISEVQLVESGGGLVQPGGSLRLSCAASGFNFWNSYIHWVRQA |
| Full heavy | | PGKGLEWVASISSYSGYTYYADSVKGRFTISADTSKNTAYLQM |
| chain | | NSLRAEDTAVYYCARSYQGQEALDYWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD1.5 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.6 | 96 | EISEVQLVESGGGLVQPGGSLRLSCAASGFNFWTAYIHWVRQA |
| Full heavy | | PGKGLEWVASISSYSGYTYYADSVKGRFTISADTSKNTAYLQM |
| chain | | NSLRAEDTAVYYCARSYQGQEALDYWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD1.6 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.7 | 97 | EISEVQLVESGGGLVQPGGSLRLSCAASGFNFFNSYIHWVRQAP |
| Full heavy | | GKGLEWVASISSYSGYTYYADSVKGRFTISADTSKNTAYLQMN |
| chain | | SLRAEDTAVYYCARSYQGQEALDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD1.7 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.8 | 98 | EISEVQLVESGGGLVQPGGSLRLSCAASGFNFHNIYLHWVRQAP |
| Full heavy | | GKGLEWVASISSYSGYTYYADSVKGRFTISADTSKNTAYLQMN |
| chain | | SLRAEDTAVYYCARSYQGQEALDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD1.8 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.9 | 99 | EISEVQLVESGGGLVQPGGSLRLSCAASGFNFHNLYVHWVRQA |
| Full heavy | | PGKGLEWVASISSYSGYTYYADSVKGRFTISADTSKNTAYLQM |
| chain | | NSLRAEDTAVYYCARSYQGQEALDYWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD1.9 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.10 | 100 | EISEVQLVESGGGLVQPGGSLRLSCAASGFNFHRAYIHWVRQAP |
| Full heavy | | GKGLEWVASISSYSGYTYYADSVKGRFTISADTSKNTAYLQMN |
| chain | | SLRAEDTAVYYCARSYQGQEALDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD1.10 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.11 | 101 | EISEVQLVESGGGLVQPGGSLRLSCAASGFNFHRIYIHWVRQAP |
| Full heavy | | GKGLEWVASISSYSGYTYYADSVKGRFTISADTSKNTAYLQMN |
| chain | | SLRAEDTAVYYCARSYQGQEALDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| sRBD1.11 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.12 | 102 | EISEVQLVESGGGLVQPGGSLRLSCAASGFNFWASYIHWVRQA |
| Full heavy | | PGKGLEWVASISSYSGYTYYADSVKGRFTISADTSKNTAYLQM |
| chain | | NSLRAEDTAVYYCARSYQGQEALDYWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD1.12 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.13 | 103 | EISEVQLVESGGGLVQPGGSLRLSCAASGFNFYASYIHWVRQAP |
| Full heavy | | GKGLEWVASISSYSGYTYYADSVKGRFTISADTSKNTAYLQMN |
| chain | | SLRAEDTAVYYCARSYQGQEALDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD1.13 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.14 | 104 | EISEVQLVESGGGLVQPGGSLRLSCAASGFNLANNYVHWVRQA |
| Full heavy | | PGKGLEWVASISSYSGYTYYADSVKGRFTISADTSKNTAYLQM |
| chain | | NSLRAEDTAVYYCARSYQGQEALDYWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD1.14 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.15 | 105 | EISEVQLVESGGGLVQPGGSLRLSCAASGFNFHNSYIHWVRQAP |
| Full heavy | | GKGLEWVASISSYSGYTYYADSVKGRFTISADTSKNTAYLQMN |
| chain | | SLRAEDTAVYYCARSYQGQEALDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD1.15 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.16 | 106 | EISEVQLVESGGGLVQPGGSLRLSCAASGFNFHRTYIHWVRQAP |
| Full heavy | | GKGLEWVASISSYSGYTYYADSVKGRFTISADTSKNTAYLQMN |
| chain | | SLRAEDTAVYYCARSYQGQEALDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD1.16 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.17 | 107 | EISEVQLVESGGGLVQPGGSLRLSCAASGFNLNNIYIHWVRQAP |
| Full heavy | | GKGLEWVASISSYSGYTYYADSVKGRFTISADTSKNTAYLQMN |
| chain | | SLRAEDTAVYYCARSYQGQEALDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD1.17 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.18 | 108 | EISEVQLVESGGGLVQPGGSLRLSCAASGFNVHWVYFHWVRQ |
| Full heavy | | APGKGLEWVASISSYSGYTYYADSVKGRFTISADTSKNTAYLQ |
| chain | | MNSLRAEDTAVYYCARSYQGQEALDYWGQGTLVTVSSASTKG |
| | | PSVFPLAPSSKSTSGGTAALGCLVKDYFPEPVTVSWNSGALTSG |
| | | VHTFPAVLQSSGLYSLSSVVTVPSSSLGTQTYICNVNHKPSNTK |
| | | VDKKVEPKSCDKTHT |
| SRBD1.18 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| SRBD1.19 | 109 | EISEVQLVESGGGLVQPGGSLRLSCAASGFNLHNQYLHWVRQA |
| Full heavy | | PGKGLEWVASISSYSGYTYYADSVKGRFTISADTSKNTAYLQM |
| chain | | NSLRAEDTAVYYCARSYQGQEALDYWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHT |
| SRBD1.19 | 77 | SDIQMTQSPSSLSASVGDRVTITCRASQSVSSAVAWYQQKPGK |
| Full light | | APKLLIYSASSLYSGVPSRFSGSRSGTDFTLTISSLQPEDFATYYC |
| chain | | QQYLYFSLVTFGQGTKVEIKRTVAAPSVFIFPPSDSQLKSGTASV |
| | | VCLLNNFYPREAKVQWKVDNALQSGNSQESVTEQDSKDSTYS |
| | | LSSTLTLSKADYEKHKVYACEVTHQGLSSPVTKSFNRGEC |
| VH FR1 | 114 | EISEVQLVESGGGLVQPGGSLRLSCAASGEN |
| VH FR2 | 115 | HWVRQAPGKGLEWVA |
| VH FR3 | 116 | YADSVKGRFTISADTSKNTAYLQMNSLRAEDTAVYYCAR |
| VH FR4 | 117 | DYWGQGTLVTVSS |
| VL FR1 | 118 | SDIQMTQSPSSLSASVGDRVTITCRASQ |
| VL FR2 | 119 | VAWYQQKPGKAPKLLIY |
| VL FR3 | 120 | GVPSRFSGSRSGTDFTLTISSLQPEDFATYYCQQ |
| VL FR4 | 121 | TFGQGTKVEIK |

TABLE 2
Summary of polypeptide SEQ ID NOs

| Poly- | VH | CDR- | VH | CDR- | VH | CDR- | VH | VL | CDR- | VL | CDR- | VL | CDR- | VL |
|---|---|---|---|---|---|---|---|---|---|---|---|---|---|---|
| peptide | FR1 | H1 | FR2 | H2 | FR3 | H3 | FR4 | FR1 | L1 | FR2 | L2 | FR3 | L3 | FR4 |
| sRBD1 | 114 | 1 | 115 | 6 | 116 | 8 | 117 | 118 | 15 | 119 | 16 | 120 | 17 | 121 |
| sRBD2 | 114 | 2 | 115 | 113 | 116 | 9 | 117 | 118 | 15 | 119 | 16 | 120 | 18 | 121 |
| sRBD3 | 114 | 3 | 115 | 113 | 116 | 10 | 117 | 118 | 15 | 119 | 16 | 120 | 19 | 121 |
| sRBD4 | 114 | 4 | 115 | 113 | 116 | 11 | 117 | 118 | 15 | 119 | 16 | 120 | 20 | 121 |
| sRBD5 | 114 | 1 | 115 | 113 | 116 | 12 | 117 | 118 | 15 | 119 | 16 | 120 | 21 | 121 |
| sRBD6 | 114 | 5 | 115 | 113 | 116 | 13 | 117 | 118 | 15 | 119 | 16 | 120 | 22 | 121 |
| sRBD7 | 114 | 1 | 115 | 7 | 116 | 14 | 117 | 118 | 15 | 119 | 16 | 120 | 23 | 121 |
| sRBD1.1 | 114 | 24 | 115 | 6 | 116 | 8 | 117 | 118 | 15 | 119 | 16 | 120 | 17 | 121 |
| sRBD1.2 | 114 | 25 | 115 | 6 | 116 | 8 | 117 | 118 | 15 | 119 | 16 | 120 | 17 | 121 |
| sRBD1.3 | 114 | 26 | 115 | 6 | 116 | 8 | 117 | 118 | 15 | 119 | 16 | 120 | 17 | 121 |
| sRBD1.4 | 114 | 27 | 115 | 6 | 116 | 8 | 117 | 118 | 15 | 119 | 16 | 120 | 17 | 121 |
| sRBD1.5 | 114 | 28 | 115 | 6 | 116 | 8 | 117 | 118 | 15 | 119 | 16 | 120 | 17 | 121 |
| sRBD1.6 | 114 | 29 | 115 | 6 | 116 | 8 | 117 | 118 | 15 | 119 | 16 | 120 | 17 | 121 |
| sRBD1.7 | 114 | 30 | 115 | 6 | 116 | 8 | 117 | 118 | 15 | 119 | 16 | 120 | 17 | 121 |
| sRBD1.8 | 114 | 31 | 115 | 6 | 116 | 8 | 117 | 118 | 15 | 119 | 16 | 120 | 17 | 121 |
| sRBD1.9 | 114 | 32 | 115 | 6 | 116 | 8 | 117 | 118 | 15 | 119 | 16 | 120 | 17 | 121 |
| sRBD1.10 | 114 | 33 | 115 | 6 | 116 | 8 | 117 | 118 | 15 | 119 | 16 | 120 | 17 | 121 |
| sRBD1.11 | 114 | 34 | 115 | 6 | 116 | 8 | 117 | 118 | 15 | 119 | 16 | 120 | 17 | 121 |
| sRBD1.12 | 114 | 35 | 115 | 6 | 116 | 8 | 117 | 118 | 15 | 119 | 16 | 120 | 17 | 121 |
| sRBD1.13 | 114 | 36 | 115 | 6 | 116 | 8 | 117 | 118 | 15 | 119 | 16 | 120 | 17 | 121 |
| sRBD1.14 | 114 | 37 | 115 | 6 | 116 | 8 | 117 | 118 | 15 | 119 | 16 | 120 | 17 | 121 |
| sRBD1.15 | 114 | 38 | 115 | 6 | 116 | 8 | 117 | 118 | 15 | 119 | 16 | 120 | 17 | 121 |
| sRBD1.16 | 114 | 39 | 115 | 6 | 116 | 8 | 117 | 118 | 15 | 119 | 16 | 120 | 17 | 121 |
| sRBD1.17 | 114 | 40 | 115 | 6 | 116 | 8 | 117 | 118 | 15 | 119 | 16 | 120 | 17 | 121 |
| sRBD1.18 | 114 | 41 | 115 | 6 | 116 | 8 | 117 | 118 | 15 | 119 | 16 | 120 | 17 | 121 |
| sRBD1.19 | 114 | 42 | 115 | 6 | 116 | 8 | 117 | 118 | 15 | 119 | 16 | 120 | 17 | 121 |

2. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

3. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within ±2 are included, in other embodiments, those which are within ±1 are included, and in still other embodiments, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein structure.

In some embodiments of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

G. Nucleic Acids

In certain embodiments, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol.

In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

1. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### II. Antibody Production

A. Antibody Production

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745 (see, e.g., Antibodies: A Laboratory Manual, Cold Spring Harbor Laboratory, 1988; incorporated herein by reference). These antibodies may be polyclonal or monoclonal antibody preparations, monospecific antisera, human antibodies, hybrid or chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof for use in various embodiments can also be synthesized in solution or on a solid support in accordance with conventional techniques. See, for example, Stewart and Young, (1984); Tarn et al, (1983); Merrifield, (1986); and Barany and Merrifield (1979), each incorporated herein by reference.

Briefly, a polyclonal antibody is prepared by immunizing an animal with an antigen or a portion thereof and collecting antisera from that immunized animal. The antigen may be altered compared to an antigen sequence found in nature. In some embodiments, a variant or altered antigenic peptide or polypeptide is employed to generate antibodies. Inocula are typically prepared by dispersing the antigenic composition in a physiologically tolerable diluent to form an aqueous composition. Antisera is subsequently collected by methods known in the arts, and the serum may be used as-is for various applications or else the desired antibody fraction may be purified by well-known methods, such as affinity chromatography (Harlow and Lane, Antibodies: A Laboratory Manual 1988).

Methods of making monoclonal antibodies are also well known in the art (Kohler and Milstein, 1975; Harlow and Lane, 1988, U.S. Pat. No. 4,196,265, herein incorporated by reference in its entirety for all purposes). Typically, this technique involves immunizing a suitable animal with a selected immunogenic composition, e.g., a purified or partially purified protein, polypeptide, peptide or domain. Resulting antibody-producing B-cells from the immunized animal, or all dissociated splenocytes, are then induced to fuse with cells from an immortalized cell line to form hybridomas. Myeloma cell lines suited for use in hybridoma-producing fusion procedures preferably are non-antibody-producing and have high fusion efficiency and enzyme deficiencies that render then incapable of growing in certain selective media that support the growth of only the desired fused cells (hybridomas). Typically, the fusion partner includes a property that allows selection of the resulting hybridomas using specific media. For example, fusion partners can be hypoxanthine/aminopterin/thymidine (HAT)-sensitive. Methods for generating hybrids of antibody-producing spleen or lymph node cells and myeloma cells usually comprise mixing somatic cells with myeloma cells in the presence of an agent or agents (chemical or electrical) that promote the fusion of cell membranes. Next, selection of hybridomas can be performed by culturing the cells by single-clone dilution in microtiter plates, followed by testing the individual clonal supernatants (after two to three weeks) for the desired reactivity. Fusion procedures for making hybridomas, immunization protocols, and techniques for isolation of immunized splenocytes for fusion are known in the art.

Other techniques for producing monoclonal antibodies include the viral or oncogenic transformation of B-lymphocytes, a molecular cloning approach may be used to generate a nucleic acid or polypeptide, the selected lymphocyte antibody method (SLAM) (see, e.g., Babcook et al., Proc. Natl. Acad. Sci. USA 93:7843-7848 (1996), the preparation of combinatorial immunoglobulin phagemid libraries from RNA isolated from the spleen of the immunized animal and selection of phagemids expressing appropriate antibodies, or producing a cell expressing an antibody from a genomic sequence of the cell comprising a modified immunoglobulin locus using Cre-mediated site-specific recombination (see, e.g., U.S. Pat. No. 6,091,001).

Monoclonal antibodies may be further purified using filtration, centrifugation, and various chromatographic methods such as HPLC or affinity chromatography. Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

The immunogenicity of a particular immunogen composition can be enhanced by the use of non-specific stimulators of the immune response, known as adjuvants. Adjuvants that may be used in accordance with embodiments include, but are not limited to, IL-1, IL-2, IL-4, IL-7, IL-12, -interferon, GMCSP, BCG, aluminum hydroxide, MDP compounds, such as thur-MDP and nor-MDP, CGP (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m2) (Johnson/Mead, NJ), cytokines such as β-interferon, IL-2, or IL-12, or genes encoding proteins involved in immune helper functions, such as B-7.A phage-display system can be used to expand antibody molecule populations in vitro. Saiki, et al., Nature 324:163 (1986); Scharf et al., Science 233:1076 (1986); U.S. Pat. Nos. 4,683,195 and 4,683,202; Yang et al., J Mol Biol. 254:392 (1995); Barbas, III et al., Methods: Comp. Meth Enzymol. (1995) 8:94; Barbas, III et al., Proc Natl Acad Sci USA 88:7978 (1991).

B. Fully Human Antibody Production

Methods are available for making fully human antibodies. Using fully human antibodies can minimize the immunogenic and allergic responses that may be caused by administering non-human monoclonal antibodies to humans as therapeutic agents. In one embodiment, human antibodies may be produced in a non-human transgenic animal, e.g., a transgenic mouse capable of producing multiple isotypes of human antibodies to protein (e.g., IgG, IgA, and/or IgE) by undergoing V-D-J recombination and isotype switching. Accordingly, this aspect applies to antibodies, antibody fragments, and pharmaceutical compositions thereof, but also non-human transgenic animals, B-cells, host cells, and hybridomas that produce monoclonal antibodies. Applications of human antibodies include, but are not limited to, detect a cell expressing an anticipated protein, either in vivo or in vitro, pharmaceutical preparations containing the antibodies of the present invention, and methods of treating disorders by administering the antibodies.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, for example, Jakobovits et al., Proc. Natl. Acad. Sci. USA 90:2551-2555 (1993); Jakobovits et al., Nature 362:255-258 (1993); Bruggermann et al., Year in Immunol. 7:33 (1993). In one example, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then crossbred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, International Patent Application Publication Nos. WO 96/33735 and WO 94/02602, which are hereby incorporated by reference in their entirety. Additional methods relating to transgenic mice for making human antibodies are described in U.S. Pat. Nos. 5,545,807; 6,713,610; 6,673,986; 6,162,963; 6,300,129; 6,255,458; 5,877,397; 5,874,299 and 5,545,806; in International Patent Application Publication Nos. WO 91/10741 and WO 90/04036; and in European Patent Nos. EP 546073B1 and EP 546073A1, all of which are hereby incorporated by reference in their entirety for all purposes.

The transgenic mice described above, referred to herein as “HuMAb” mice, contain a human immunoglobulin gene minilocus that encodes unrearranged human heavy (μ and γ) and κ light chain immunoglobulin sequences, together with targeted mutations that inactivate the endogenous μ and κ chain loci (Lonberg et al., Nature 368:856-859 (1994)). Accordingly, the mice exhibit reduced expression of mouse IgM or κ chains and in response to immunization, the introduced human heavy and light chain transgenes undergo class switching and somatic mutation to generate high affinity human IgG κ monoclonal antibodies (Lonberg et al., supra; Lonberg and Huszar, Intern. Ref. Immunol. 13:65-93 (1995); Harding and Lonberg, Ann. N.Y. Acad. Sci. 764:536-546 (1995)). The preparation of HuMAb mice is described in detail in Taylor et al., Nucl. Acids Res. 20:6287-6295 (1992); Chen et al., Int. Immunol. 5:647-656 (1993); Tuaillon et al., J. Immunol. 152:2912-2920 (1994); Lonberg et al., supra; Lonberg, Handbook of Exp. Pharmacol. 113:49-101 (1994); Taylor et al., Int. Immunol. 6:579-591 (1994); Lonberg and Huszar, Intern. Ref. Immunol. 13:65-93 (1995); Harding and Lonberg, Ann. N.Y. Acad. Sci. 764:536-546 (1995); Fishwild et al., Nat. Biotechnol. 14:845-851 (1996); the foregoing references are herein incorporated by reference in their entirety for all purposes. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807; as well as International Patent Application Publication Nos. WO 93/1227; WO 92/22646; and WO 92/03918, the disclosures of all of which are hereby incorporated by reference in their entirety for all purposes. Technologies utilized for producing human antibodies in these transgenic mice are disclosed also in WO 98/24893, and Mendez et al., Nat. Genetics 15:146-156 (1997), which are herein incorporated by reference. For example, the HCo7 and HCo12 transgenic mice strains can be used to generate human antibodies.

Using hybridoma technology, antigen-specific humanized monoclonal antibodies with the desired specificity can be produced and selected from the transgenic mice such as those described above. Such antibodies may be cloned and expressed using a suitable vector and host cell, or the antibodies can be harvested from cultured hybridoma cells. Fully human antibodies can also be derived from phage-display libraries (as disclosed in Hoogenboom et al., J. Mol. Biol. 227:381 (1991); and Marks et al., J. Mol. Biol. 222:581 (1991)). One such technique is described in International Patent Application Publication No. WO 99/10494 (herein incorporated by reference), which describes the isolation of high affinity and functional agonistic antibodies for 1V1PL- and msk-receptors using such an approach.

C. Antibody Fragments Production

Antibody fragments that retain the ability to recognize the antigen of interest will also find use herein. A number of antibody fragments are known in the art that comprise antigen-binding sites capable of exhibiting immunological binding properties of an intact antibody molecule and can be subsequently modified by methods known in the arts. Functional fragments, including only the variable regions of the heavy and light chains, can also be produced using standard techniques such as recombinant production or preferential proteolytic cleavage of immunoglobulin molecules. These fragments are known as Fv. See, e.g., Inbar et al., Proc. Nat. Acad. Sci. USA 69:2659-2662 (1972); Hochman et al., Biochem. 15:2706-2710 (1976); and Ehrlich et al., Biochem. 19:4091-4096 (1980).

Single-chain variable fragments (scFvs) may be prepared by fusing DNA encoding a peptide linker between DNAs encoding the two variable domain polypeptides (VL and VH). scFvs can form antigen-binding monomers, or they can form multimers (e.g., dimers, trimers, or tetramers), depending on the length of a flexible linker between the two variable domains (Kortt et al., Prot. Eng. 10:423 (1997); Kort et al., Biomol. Eng. 18:95-108 (2001)). By combining different VL- and VH-comprising polypeptides, one can form multimeric scFvs that bind to different epitopes (Kriangkum et al., Biomol. Eng. 18:31-40 (2001)). Antigen-binding fragments are typically produced by recombinant DNA methods known to those skilled in the art. Although the two domains of the Fv fragment, VL and VH, are coded for by separate genes, they can be joined using recombinant methods by a synthetic linker that enables them to be made as a single chain polypeptide (known as single chain Fv (sFv or scFv); see e.g., Bird et al., Science 242:423-426 (1988); and Huston et al., Proc. Natl. Acad. Sci. USA 85:5879-5883 (1988). Design criteria include determining the appropriate length to span the distance between the C-terminus of one chain and the N-terminus of the other, wherein the linker is generally formed from small hydrophilic amino acid residues that do not tend to coil or form secondary structures. Suitable linkers generally comprise polypeptide chains of alternating sets of glycine and serine residues, and may include glutamic acid and lysine residues inserted to enhance solubility. Antigen-binding fragments are screened for utility in the same manner as intact antibodies. Such fragments include those obtained by amino-terminal and/or carboxy-terminal deletions, where the remaining amino acid sequence is substantially identical to the corresponding positions in the naturally occurring sequence deduced, for example, from a full-length cDNA sequence.

Antibodies may also be generated using peptide analogs of the epitopic determinants disclosed herein, which may consist of non-peptide compounds having properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al., J. Med. Chem. 30:1229 (1987). Liu et al. (2003) also describe “antibody like binding peptidomimetics” (ABiPs), which are peptides that act as pared-down antibodies and have certain advantages of longer serum half-life as well as less cumbersome synthesis methods. These analogs can be peptides, non-peptides or combinations of peptide and non-peptide regions. Fauchere, Adv. Drug Res. 15:29 (1986); Veber and Freidiner, TINS p. 392 (1985); and Evans et al., J. Med. Chem. 30:1229 (1987), which are incorporated herein by reference in their entirety for any purpose. Peptide mimetics that are structurally similar to therapeutically useful peptides may be used to produce a similar therapeutic or prophylactic effect. Such compounds are often developed with the aid of computerized molecular modeling. Generally, peptidomimetics of the invention are proteins that are structurally similar to an antibody displaying a desired biological activity, such as the ability to bind a protein, but have one or more peptide linkages optionally replaced by a linkage selected from: —CH2NH—, —CH2S—, —CH2-CH2-, —CH═CH— (cis and trans), COCH2-, —CH(OH)CH2-, and —CH2SO— by methods well known in the art. Systematic substitution of one or more amino acids of a consensus sequence with a D-amino acid of the same type (e.g., D-lysine in place of L-lysine) may be used in certain embodiments of the invention to generate more stable proteins. In addition, constrained peptides comprising a consensus sequence or a substantially identical consensus sequence variation may be generated by methods known in the art (Rizo and Gierasch, Ann. Rev. Biochem. 61:387 (1992), incorporated herein by reference), for example, by adding internal cysteine residues capable of forming intramolecular disulfide bridges which cyclize the peptide.

Once generated, a phage display library can be used to improve the immunological binding affinity of the Fab molecules using known techniques. See, e.g., Figini et al., J. Mol. Biol. 239:68 (1994). The coding sequences for the heavy and light chain portions of the Fab molecules selected from the phage display library can be isolated or synthesized and cloned into any suitable vector or replicon for expression. Any suitable expression system can be used.

### III. Obtaining Encoded Antibodies

In some aspects, there are nucleic acid molecule encoding antibody polypeptides (e.g., heavy or light chain, variable domain only, or full-length). These may be generated by methods known in the art, e.g., isolated from B cells of mice that have been immunized and isolated, phage display, expressed in any suitable recombinant expression system and allowed to assemble to form antibody molecules.

A. Expression

The nucleic acid molecules may be used to express large quantities of recombinant antibodies or to produce chimeric antibodies, single chain antibodies, immunoadhesins, diabodies, mutated antibodies, and other antibody derivatives. If the nucleic acid molecules are derived from a non-human, non-transgenic animal, the nucleic acid molecules may be used for antibody humanization.

1. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the antibodies, or antigen-binding fragments thereof, DNAs encoding partial or full-length light and heavy chains are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

2. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

3. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

4. Host Cells

In another aspect, contemplated are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to T-cell activation, such as one that is controlled by NFAT-1 or NF-κB, both of which are transcription factors that can be activated upon T-cell activation. Control of antibody expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

B. Isolation

The nucleic acid molecule encoding either or both of the entire heavy and light chains of an antibody or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. See, e.g., Kabat et al., 1991, supra. Nucleic acid molecules encoding the full-length heavy and/or light chains may then be expressed in a cell into which they have been introduced and the antibody isolated.

### IV. Viruses

Aspects of the present disclosure relate to treatment, analysis, or use of a virus. In some embodiments, disclosed are methods for treatment or prevention of a viral infection. In some embodiments, disclosed are compositions comprising one or more anti-viral agents. In some embodiments, disclosed are methods for diagnosis of a viral infection. In some embodiments, disclosed are methods for detection of a virus in a sample.

A. Coronaviruses

In particular embodiments, the virus is from the family Coronaviridae. Coronaviridae is a family of enveloped, positive-sense, single-stranded RNA viruses. Coronavirus is the common name for Coronaviridae and Orthocoronavirinae (also referred to as Coronavirinae). The family Coronaviridae is organized in 2 sub-families, 5 genera, 23 sub-genera and approximately 40 species. They are enveloped viruses having a positive-sense single-stranded RNA genome and a nucleocapsid having helical symmetry. The genome size of coronaviruses ranges from approximately 26-32 kilobases.

The present disclosure encompasses treatment or prevention of infection of any virus in the Coronaviridae family. In certain embodiments, the disclosure encompasses treatment or prevention of infection of any virus in the subfamily Coronavirinae and including the four genera, Alpha-, Beta-, Gamma-, and Deltacoronavirus. In specific embodiments, the disclosure encompasses treatment or prevention of infection of any virus in the genus of Betacoronavirus, including the subgenus Sarbecovirus and including the species of severe acute respiratory syndrome-related coronavirus. In specific embodiments, the disclosure encompasses treatment or prevention of infection of any virus in the species of severe acute respiratory syndrome-related coronavirus, including the strains severe acute respiratory syndrome coronavirus (SARS-CoV) and severe acute respiratory syndrome coronavirus 2 (SARS-CoV-2, the virus that causes COVID-19). The disclosure encompasses treatment or prevention of infection any isolate, strain, type (including Type A, Type B and Type C; Forster et al., 2020, PNAS, available on the World Wide Web at doi.org/10.1073/pnas.2004999117), cluster, or sub-cluster of the species of severe acute respiratory syndrome-related coronavirus, including at least SARS-CoV-2. In specific embodiments, the virus has a genome length between 29000 to 30000, between 29100 and 29900, between 29200 and 29900, between 29300 and 29900, between 29400 and 29900, between 29500 and 29900, between 29600 and 29900, between 29700 and 29900, between 29800 and 29900, or between 29780 and 29900 base pairs in length.

Examples of specific SARS-CoV-2 viruses include the following listed in the NCBI GenBank® Database, and these GenBank® Accession sequences are incorporated by reference herein in their entirety: (a) LC534419 and LC534418 and LC528233 and LC529905 (examples of different strains from Japan); (b) MT281577 and MT226610 and NC_045512 and MN996531 and MN908947 (examples of different strains from China); (c) MT281530 (Iran); (d) MT126808 (Brazil); (e) MT020781 (Finland); (f) MT093571 (Sweden); (g) MT263074 (Peru); (h) MT292582 and MT292581 and MT292580 and MT292579 (examples of different strains from Spain); (i) examples from the United States, such as MT276331 (TX); MT276330 (FL); MT276328 (OR) MT276327 (GA); MT276325 (WA); MT276324 (CA); MT276323 (RI); MT188341 (MN); and (j) MT276598 (Israel). In particular embodiments, the disclosure encompasses treatment or prevention of infection of any of these or similar viruses, including viruses whose genome has at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. In particular embodiments, the disclosure encompasses treatment or prevention of infection of any of these or similar viruses, including viruses whose genome has its entire sequence that is greater than 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. As one specific example, the present disclosure includes methods of treatment or prevention of infection of a virus having a genome sequence of SEQ ID NO:110 (represented by GenBank® Accession No. NC_045512; origin Wuhan, China) and any virus having a genome sequence with at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to SEQ ID NO:110.

Aspects of the disclosure relate to polypeptides that bind to a SARS-CoV-2 spike (S) protein. In some embodiments, disclosed are polypeptides that specifically bind to a receptor binding domain (RBD) of a SARS-CoV-2 S protein. Sequences of SARS-CoV-2 S protein and RBD are provided in Table 2 below.

TABLE 2

| | SEQ ID | |
|---|---|---|
| Name | NO | Sequence |
| SARS-CoV-2 | 111 | MFVFLVLLPLVSSQCVNLTT |
| Spike (S) | | RTQLPPAYTNSFTRGVYYPD |
| Protein | | KVFRSSVLHSTQDLFLPFFS |
| | | NVTWFHAIHVSGTNGTKRFD |
| | | NPVLPFNDGVYFASTEKSNI |
| | | IRGWIFGTTLDSKTQSLLIV |
| | | NNATNVVIKVCEFQFCNDPF |
| | | LGVYYHKNNKSWMESEFRVY |
| | | SSANNCTFEYVSQPFLMDLE |
| | | GKQGNFKNLREFVFKNIDGY |
| | | FKIYSKHTPINLVRDLPQGF |
| | | SALEPLVDLPIGINITRFQT |
| | | LLALHRSYLTPGDSSSGWTA |
| | | GAAAYYVGYLQPRTFLLKYN |
| | | ENGTITDAVDCALDPLSETK |
| | | CTLKSFTVEKGIYQTSNFRV |
| | | QPTESIVRFPNITNLCPFGE |
| | | VENATRFASVYAWNRKRISN |
| | | CVADYSVLYNSASFSTFKCY |
| | | GVSPTKLNDLCFTNVYADSF |
| | | VIRGDEVRQIAPGQTGKIAD |
| | | YNYKLPDDFTGCVIAWNSNN |
| | | LDSKVGGNYNYLYRLFRKSN |
| | | LKPFERDISTEIYQAGSTPC |
| | | NGVEGFNCYFPLQSYGFQPT |
| | | NGVGYQPYRVVVLSFELLHA |
| | | PATVCGPKKSTNLVKNKCVN |
| | | FNFNGLTGTGVLTESNKKFL |
| | | PFQQFGRDIADTTDA VRDP |
| | | QTLEILDITPCSFGGVSVIT |
| | | PGTNTSNQVAVLYQDVNCTE |
| | | VPVAIHADQLTPTWRVYSTG |
| | | SNVFQTRAGCLIGAEHVNNS |
| | | YECDIPIGAGICASYQTQTN |
| | | SPRRARSVASQSIIAYTMSL |
| | | GAENSVAYSNNSIAIPTNFT |
| | | ISVTTEILPVSMTKTSVDCT |
| | | MYICGDSTECSNLLLQYGSF |
| | | CTQLNRALTGIAVEQDKNTQ |
| | | EVFAQVKQIYKTPPIKDFGG |
| | | FNFSQILPDPSKPSKRSFIE |
| | | DLLFNKVTLADAGFIKQYGD |
| | | CLGDIAARDLICAQKFNGLT |
| | | VLPPLLTDEMIAQYTSALLA |
| | | GTITSGWTFGAGAALQIPFA |
| | | MQMAYRENGIGVTQNVLYEN |
| | | QKLIANQFNSAIGKIQDSLS |
| | | STASALGKLQDVVNQNAQAL |
| | | NTLVKQLSSNFGAISSVLND |
| | | ILSRLDKVEAEVQIDRLITG |
| | | RLQSLQTYVTQQLIRAAEIR |
| | | ASANLAATKMSECVLGQSKR |
| | | VDFCGKGYHLMSFPQSAPHG |
| | | VVFLHVTYVPAQEKNFTTAP |
| | | AICHDGKAHFPREGVFVSNG |
| | | THWFVTQRNFYEPQIITTDN |
| | | TFVSGNCDVVIGIVNNTVYD |
| | | PLQPELDSFKEELDKYFKNH |
| | | TSPDVDLGDISGINASVVNI |
| | | QKEIDRLNEVAKNLNESLID |
| | | LQELGKYEQYIKWPWYIWLG |
| | | FIAGLIAIVMVTIMLCCMTS |
| | | CCSCLKGCCSCGSCCKFDED |
| | | DSEPVLKGVKLHYT |
| Receptor | 112 | RVQPTESIVRFPNITNLCPF |
| Binding | | GEVFNATRFASVYAWNRKRI |
| Domain | | SNCVADYSVLYNSASFSTFK |
| (RBD) of | | CYGVSPTKLNDLCFTNVYAD |
| SARS-CoV-2 | | SFVIRGDEVRQIAPGQTGKI |
| S protein | | ADYNYKLPDDFTGCVIAWNS |
| | | NNLDSKVGGNYNYLYRLFRK |
| | | SNLKPFERDISTEIYQAGST |
| | | PCNGVEGENCYFPLQSYGFQ |
| | | PTNGVGYQPYRVVVLSFELL |
| | | HAPATVCGPKKSTNLVKNKC |
| | | VNF |

SARS-CoV-2 proteins, including SARS-CoV-2 spike protein, are described in detail in, for example, Yoshimoto F. K. (2020). The protein journal, 39(3), 198-216, incorporated herein by reference in its entirety.

### V. Pharmaceutical Compositions

The present disclosure includes methods for treating disease and modulating immune responses in a subject in need thereof. The disclosure includes cells that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection. In some embodiments, compositions of the present disclosure (e.g., compositions comprising SARS-CoV-2 spike protein-binding polypeptides) are administered to a subject intravenously.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most or at least 3, 4, 5, 6, 7, 8, 9, 10 or more. The administrations may range from 2-day to 12-week intervals, more usually from one to two week intervals.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

Pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (e.g., polypeptides of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of the immune checkpoint inhibitor, such as an antibody and/or microbial modulator, that is administered to a human will be in the range of about 0.01 to about 50 mg/kg of patient body weight whether by one or more administrations. In some embodiments, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

VI. Detection pairs

Detection pairs include two complementary proteins, nucleic acids, or molecules that upon interaction produces a readout such as an enzymatic activity or colorimetric or fluorescent signal. Protein detection pairs can be two halves of an enzyme that upon interaction become one active enzyme. Enzymes include beta-lactamase, dihydrofolate reductase, focal adhesion kinase, horseradish peroxidase, Gal4, beta-galactosidase, luciferase or tobacco etch virus protease. Protein detection pairs can also be two halves of a fluorescent protein that upon interaction produce a fluorescence signal. Detection pairs can also comprise fluorophores or chromophores which involve a donor and acceptor whose proximity generates a detectable signal of fluorescence of phosphorescence.

### VII. Detectable Labels

In some aspects of this disclosure, it will be useful to detectably or therapeutically label a Fab polypeptide or protein G Fab-binding domain. Methods for conjugating polypeptides to these agents are known in the art. For the purpose of illustration only, polypeptides can be labeled with a detectable moiety such as a radioactive atom, a chromophore, a fluorophore, or the like. Such labeled polypeptides can be used for diagnostic techniques, either in vivo, or in an isolated test sample or in methods described herein.

As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition that is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6.sup.th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6.sup.th ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/polypeptides, e.g., rhodamine/anti-rhodamine, biotin/avidin and biotin/strepavidin. The coupling of polypeptides to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten polypeptides. See, Harlow and Lane (1988) supra.

### VIII. Kits

Certain aspects of the present invention also concern kits containing compositions of the disclosure or compositions to implement methods of the disclosure. In some embodiments, kits can be used to detect the presence of a SARS-CoV-2 virus in a sample. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, a kit contains one or more polypeptides capable of binding to a SARS-CoV-2 spike protein, including polypeptides disclosed herein. For example, a kit may comprise at least 1, 2, 3, 4, 5, 6, 7, 8, 9, 10 or more Fabs disclosed herein for detecting a SARS-CoV-2 spike protein. In some embodiments, a kit comprises a detection pair. In some embodiments, a kit comprises an enzyme. In some embodiments, a kit comprises a substrate for an enzyme.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. In certain aspects, negative and/or positive control nucleic acids, probes, and inhibitors are included in some kit embodiments.

Kits may further comprise instructions for use. For example, in some embodiments, a kit comprises instructions for detecting a SARS-CoV-2 virus in a sample.

It is contemplated that any method or composition described herein can be implemented with respect to any other method or composition described herein and that different embodiments may be combined. The claims originally filed are contemplated to cover claims that are multiply dependent on any filed claim or combination of filed claims.

### EXAMPLES

The following examples are included to demonstrate certain embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1—Generation of Synthetic Antibodies Recognizing SARS-CoV-2 RBD by Phage Display Mutagenesis

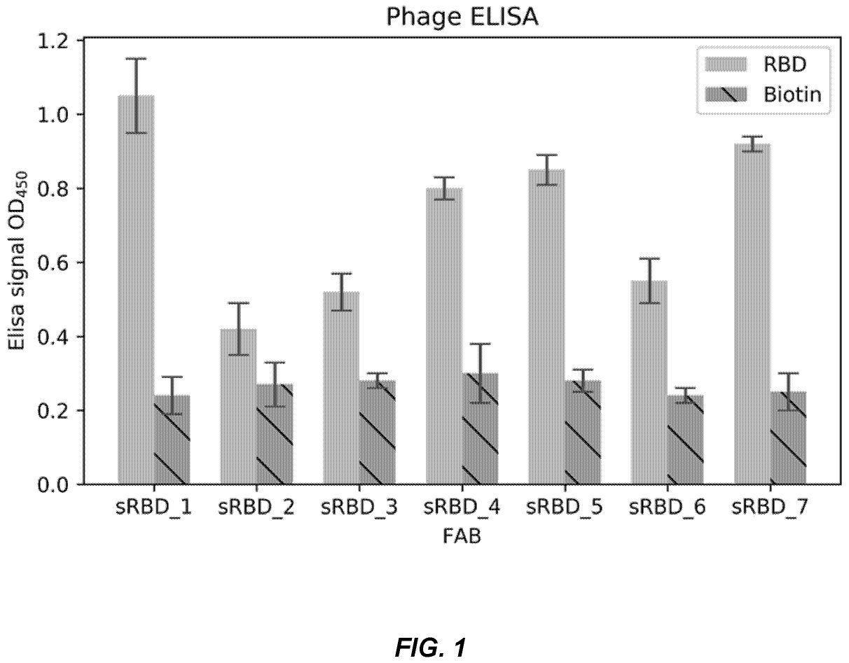

The receptor binding domain (RBD) of the SARS-CoV-2 spike protein was expressed in mammalian cells. The RBD was biotinylated via glycoproteins with EZ-Link Hydrazide Biotins (Thermo) to facilitate immobilization onto streptavidin-coated paramagnetic beads. Five rounds of phage display selection were performed with a high diversity (10¹⁰) synthetic library¹ using previously published protocols^{2; 3}. To obtain high affinity binders, the concentration of the RBD antigen was systematically reduced, starting at 200 nM during the first round and ending with 1 nM in round 5. To acquire binders with a slow dissociation rate, additional selection pressure was applied by using longer washes during panning. Phage ELISA was performed on 192 clones, resulting in identifying 5 unique high affinity variants, named sRBD1, sRBD2, sRBD3, sRBD4, and sRBD5. The highest ELISA signal was observed for sRBD1 (FIG. 1). Next, a second biopanning campaign was performed to obtain Fabs with binding epitopes that were non-overlapping to that of the sRBD1 antibody. Having the high affinity sRBD1 Fab in hand from the first selection, the inventors sought to generate a second set of Fabs that bound to the RBD through independent epitopes. To obtain these Fabs, an epitope masking strategy was employed that involved adding the RBD1 Fab as a competitor during a second biopanning campaign. To do so, five rounds of selection with sRBD1 present as a soluble competitor were performed starting with 200 nM antigen concentration in a round one and ending with 1 nM in round 5. Positive clones after phage ELISA were sequenced and 2 unique sequences were found: sRBD6 and sRBD7, as well as previously selected sRBD5. Sequences for sRBD1-sRBD7 are provided in Table 3 below.

TABLE 3

| Fab | CDR-L1 | CDR-L2 | CDR-L3 | CDR-H1 | CDR-H2 | CDR-H3 |
|---|---|---|---|---|---|---|
| SRBD1 | SVSSA | SASSLYS | YLYFSL V | FSSSYI | SISSYSGYTY | SYQGQEAL |
| | (SEQ ID | (SEQ ID | (SEQ ID | (SEQ ID | (SEQ ID | (SEQ ID |
| | NO: 15) | NO: 16) | NO: 17) | NO: 1) | NO: 6) | NO: 8) |
| SRBD2 | SVSSA | SASSLYS | SNYSYPI | VYSSYI | YISSYYGSTY | YAGHAM |
| | (SEQ ID | (SEQ ID | (SEQ ID | (SEQ ID | (SEQ ID | (SEQ ID |
| | NO: 15) | NO: 16) | NO: 18) | NO: 2) | NO: 113) | NO: 9) |
| SRBD3 | SVSSA | SASSLYS | ASWGSPI | IYSSYI | YISSYYGSTY | YGDDAM |
| | (SEQ ID | (SEQ ID | (SEQ ID | (SEQ ID | (SEQ ID | (SEQ ID |
| | NO: 15) | NO: 16) | NO: 19) | NO: 3) | NO: 113) | NO: 10) |
| SRBD4 | SVSSA | SASSLYS | SYYGSLI | LYSSYI | YISSYYGSTY | YHSWAF |
| | (SEQ ID | (SEQ ID | (SEQ ID | (SEQ ID | (SEQ ID | (SEQ ID |
| | NO: 15) | NO: 16) | NO: 20) | NO: 4) | NO: 113) | NO: 11) |
| SRBD5 | SVSSA | SASSLYS | SYYGNLI | VYSSYI | YISSYYGSTY | YSYPGGM |
| | (SEQ ID | (SEQ ID | (SEQ ID | (SEQ ID | (SEQ ID | (SEQ ID |
| | NO: 15) | NO: 16) | NO: 21) | NO: 2) | NO: 113) | NO: 12) |
| SRBD6 | SVSSA | SASSLYS | HAWGYLI | VSSSYI | YISSYYGSTY | YAEGMGM |
| | (SEQ ID | (SEQ ID | (SEQ ID | (SEQ ID | (SEQ ID | (SEQ ID |
| | NO: 15) | NO: 16) | NO: 22) | NO: 5) | NO: 113) | NO: 13) |
| SRBD7 | SVSSA | SASSLYS | YYYGALI | VYSSYI | YISPYYGSTY | YWSYGGM |
| | (SEQ ID | (SEQ ID | (SEQ ID | (SEQ ID | (SEQ ID | (SEQ ID |
| | NO: 15) | NO: 16) | NO: 23) | NO: 2) | NO: 7) | NO: 14) |

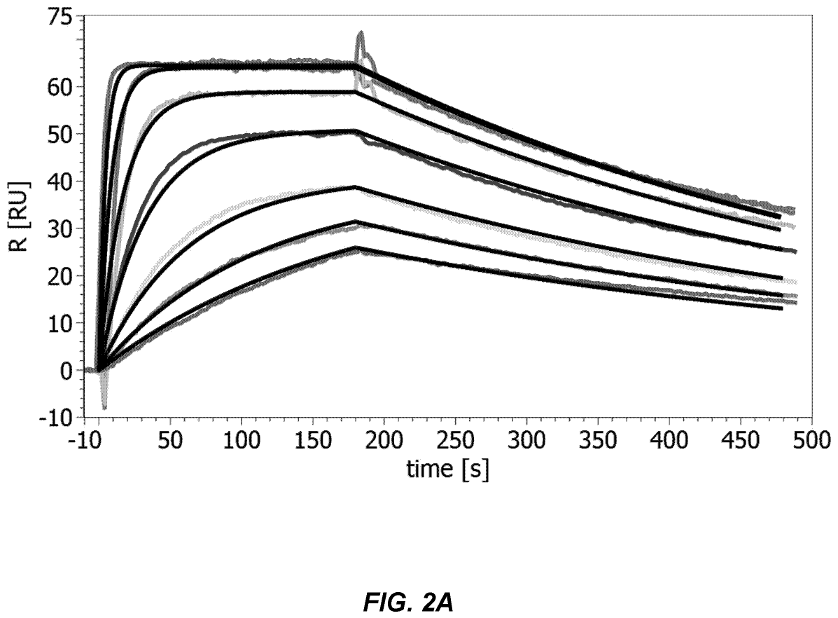

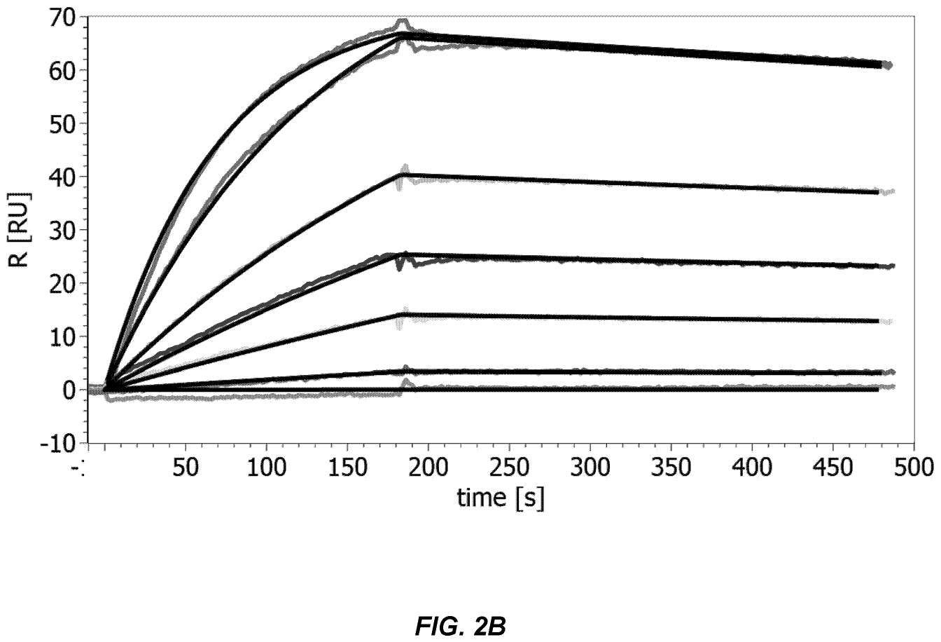

The two non-overlapping Fabs with the highest phage ELISA signal (sRBD1 and sRBD7) were chosen for further characterization and their binding kinetics affinities were determined by Surface Plasmon Resonance (SPR). Both Fabs had a similar binding constant (K_{D}), at 3 nM for sRBD1 and 2.5 nM for sRBD7, but differed in their respective association and dissociation rates. sRBD7 has an order of magnitude slower dissociation rate (K_{off}) compared with sRBD1 (FIGS. 2A-2B, Table 4).

TABLE 4

| Fab | Kₒₙ (M⁻¹ s⁻¹) | K_{off} (s⁻¹) | K_{D} (nM) | χ² (RU²) |
|---|---|---|---|---|
| sRBD1 | 4.65 × 10⁵ | 1.38 × 10⁻³ | 2.98 | 2.19 |
| sRBD7 | 1.5 × 10⁵ | 2.9 × 10⁻⁴ | 2.55 | 0.49 |

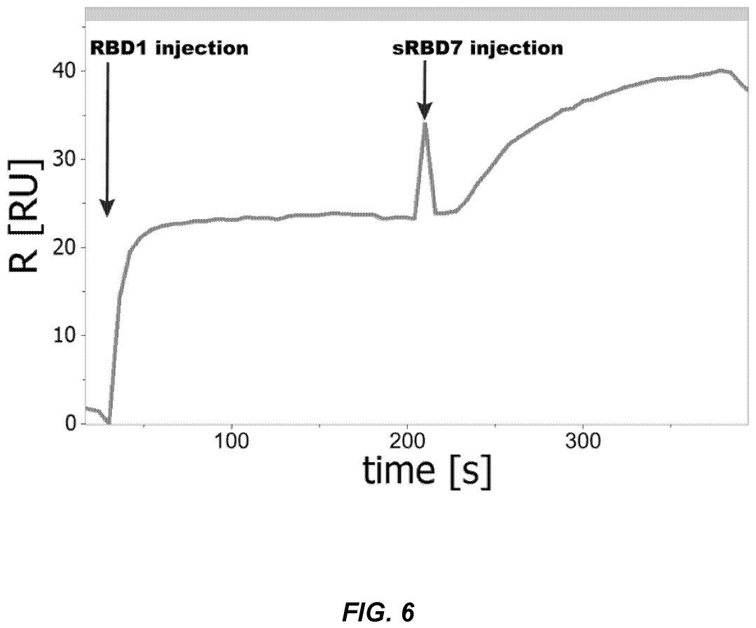

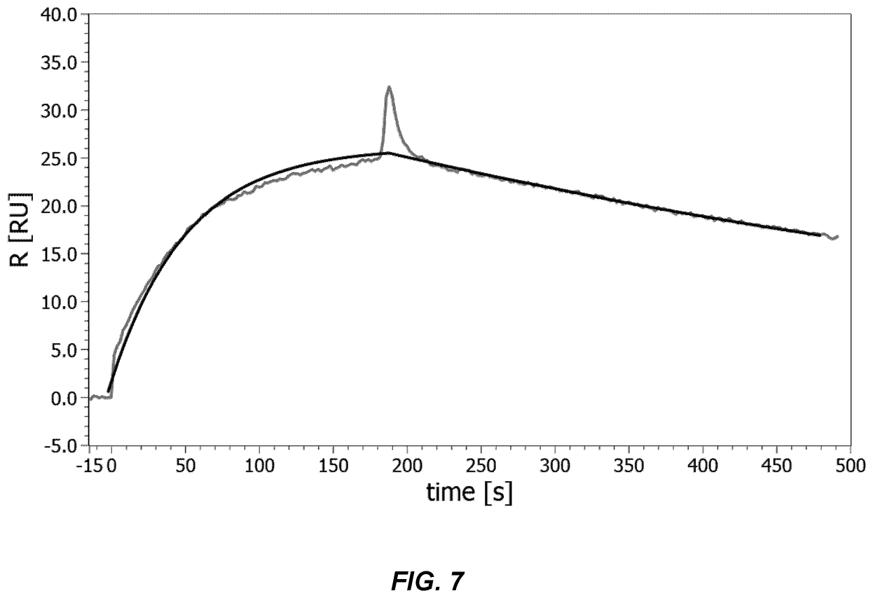

To assess the diversity of the epitope, epitope binning experiments were performed by SPR. This revealed that sRBD1 and sRBD7 bound to two distinct epitopes (FIG. 6). sRBD1 binding to the full SARS-CoV-2 Spike protein was validated by single injection SPR (FIG. 7), which showed a similar binding profile to that observed for its binding to the RBD.

### Example 2—Antibody-Mediated SARS-CoV-2 Neutralization

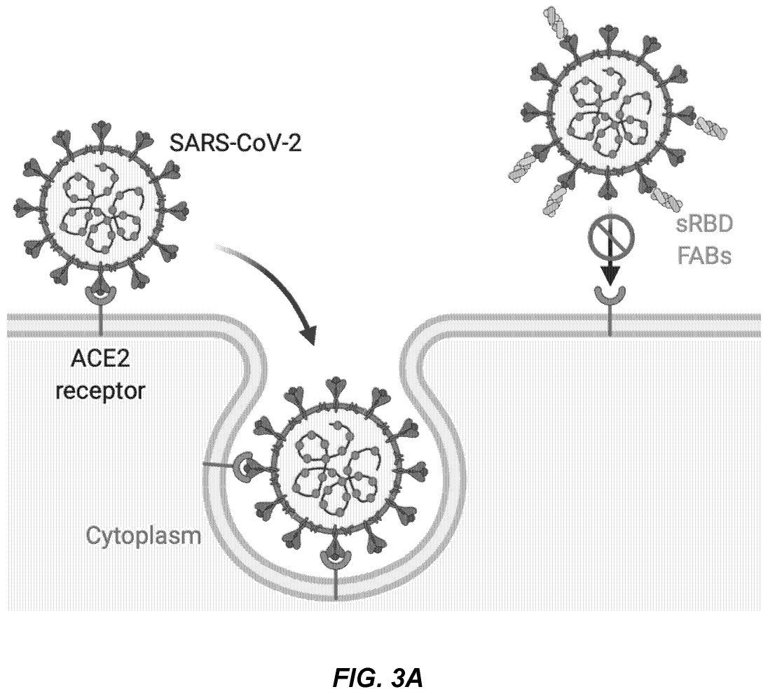

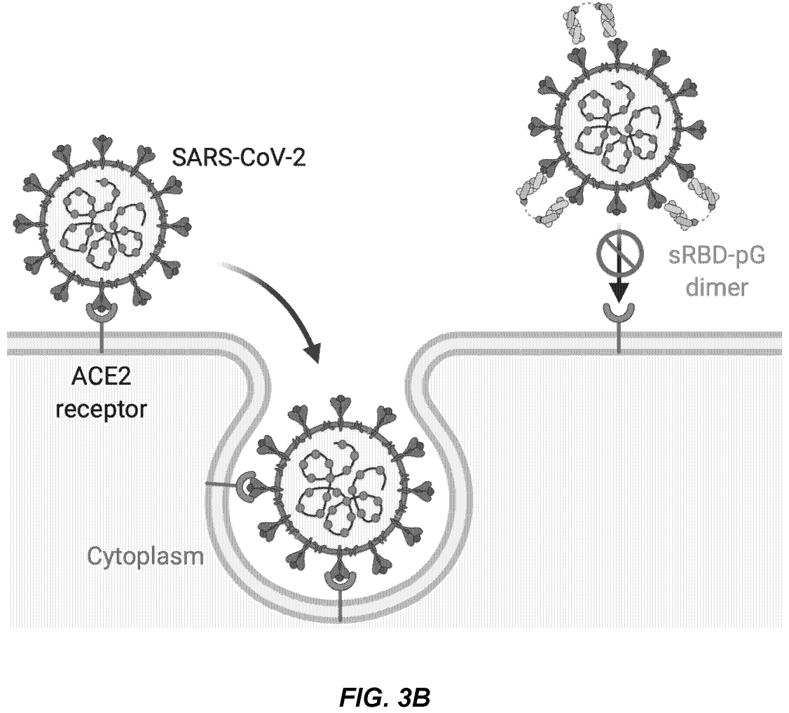

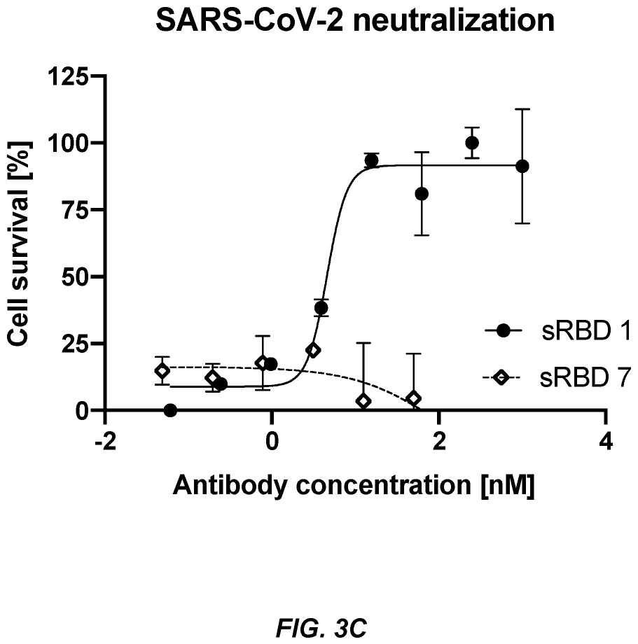

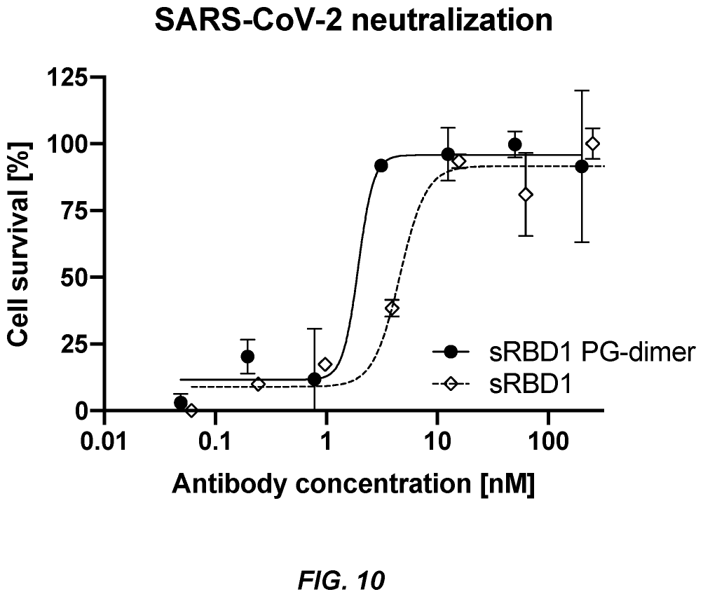

To determine the level of virus neutralization, the Fabs were tested in a plaque reduction assay that measured the cell survival afforded by the Fabs. sRBD1 showed dose-dependent SARS-CoV-2 infection neutralization with the 50% neutralization at 4.8 nM (FIG. 3C). These results revealed high potential for anti-viral efficacy. sRBD7 did not show any virus neutralization capabilities (FIG. 3C), consistent with recognition of a different epitope than sRBD1. In an attempt to mimic the avidity effect, the inventors tested a Protein G-A1 dimer with 5 and 52 Gly-Ser linker, as well as a trimer premixed with LRT grafted 4 sRBD1 fab. The neutralization values were 2.5-times improved relative to sRBD1 alone (FIG. 10).

### Example 3—Affinity Maturation of sRBD1

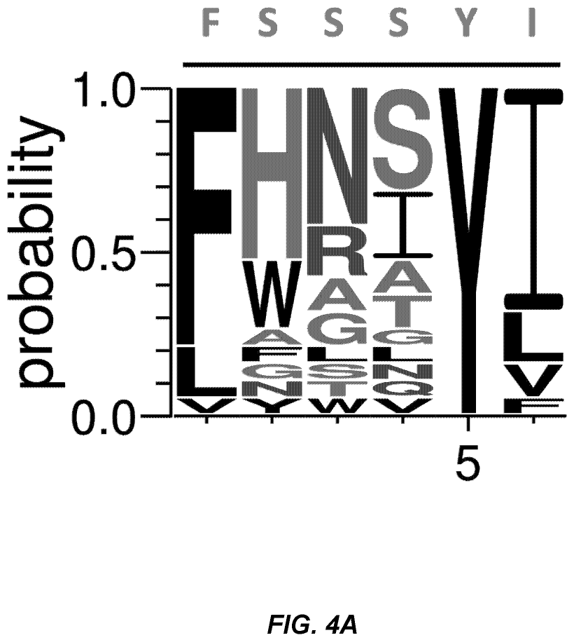

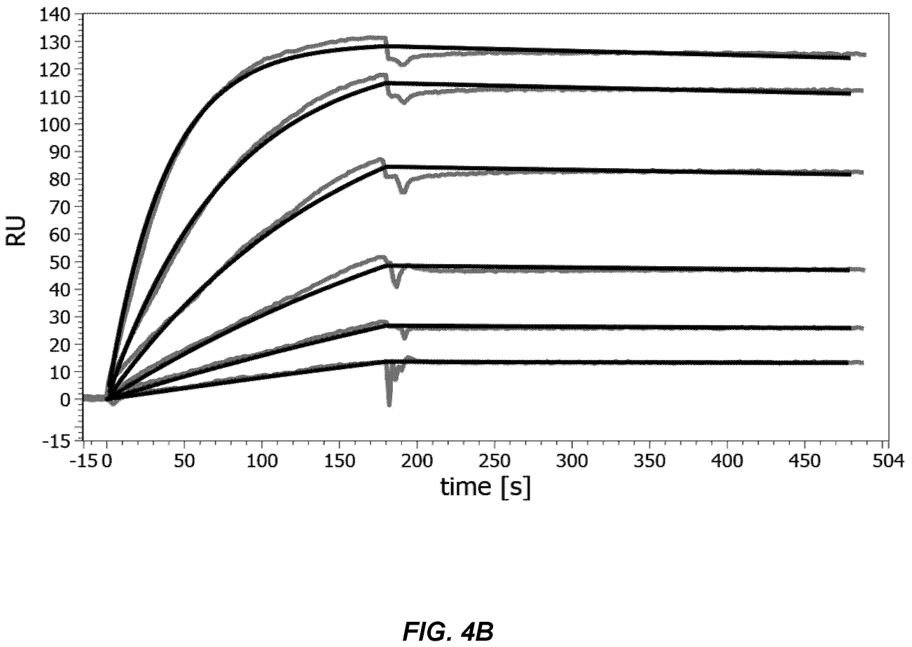

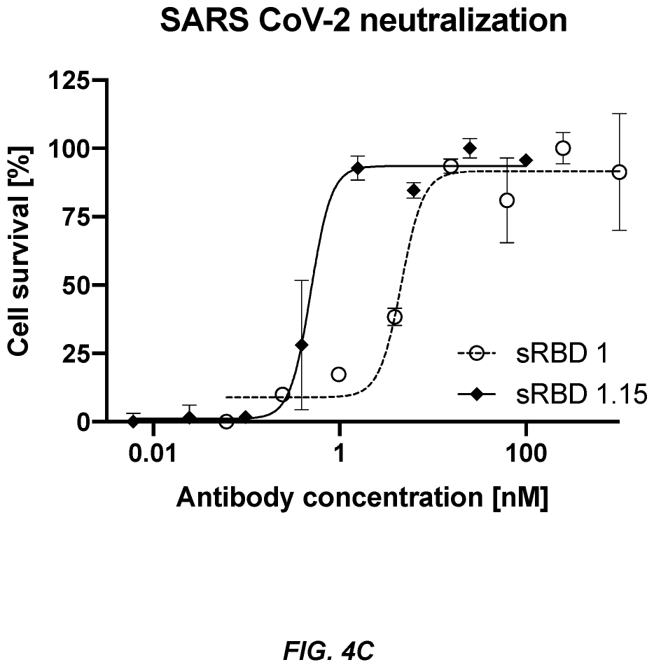

The inventors initiated an affinity maturation campaign by generating phage display libraries to introduce higher diversity in CDR-H1, using a combination of “hard” and “tailored” randomization strategies. To that end, two sub-libraries were generated, where the first library was biased to hydrophobic and aromatic amino acids, while in the second library, hard randomization was used to introduce all possible amino acids. To ensure the slower off-rate kinetics of Fabs, additional long-washes selection pressure was applied during the biopanning. In this strategy, three rounds of selection were performed with the RBD concentration decreased from 10 nM to 20 pM. Each round was additionally supplemented with four consecutive 30 minute washing steps to promote slow dissociation of selected candidates. After round 3, 96 clones were picked and tested in phage ELISA leading to the identification of 19 sequence unique Fabs. Sequence alignment of these 19 unique CDR-H1 variants shows no randomization at position 1 and a very limited randomization at position 5 and 6 of CDR-H1 compared to original sRBD1 CDR-H1. High sequence variability at position 2, 3 and 4 with a preference for His at position 2 was observed (FIG. 4A). The binding kinetics generally showed 10-30-fold affinity improvement of matured CDR-H1 variants (Table 5). SPR kinetics of the highest affinity variant, sRBD1.5, showed an affinity of 42 pM (Table 5). SPR kinetics of sRBD1.15 showed an affinity (KD) of 100 pM, and both association and dissociation rates were improved by order of magnitude compared to WT sRBD1 (Table 6 and FIG. 4B). Additionally, the affinity matured Fab sRBD1.15 had over 10-times improved neutralization capabilities (IC₅₀=0.48 nM) in a monovalent form (FIG. 4C).

TABLE 5

| Fab | CDR-H1 Variant | Kₒₙ (M⁻¹ s⁻¹) | K_{off} (s⁻¹) | K_{D} (nM) |
|---|---|---|---|---|
| SRBD1.1 | FGGSYI (SEQ ID | 5.70 × 10⁵ | 3.05 × 10⁻⁴ | 0.536 |
| | NO: 24) | | | |
| SRBD1.2 | FHGIYL (SEQ ID | 9.62 × 10⁵ | 2.69 × 10⁻⁴ | 0.279 |
| | NO: 25) | | | |
| SRBD1.3 | FHSTYI (SEQ ID | 7.92 × 10⁵ | 3.57 × 10⁻⁴ | 0.449 |
| | NO: 26) | | | |
| SRBD1.4 | FWLGYI (SEQ ID | 8.90 × 10⁵ | 2.29 × 10⁻⁴ | 0.256 |
| | NO: 27) | | | |
| SRBD1.5 | FWNSYI (SEQ ID | 7.11 × 10⁵ | 2.98 × 10⁻⁵ | 0.042 |
| | NO: 28) | | | |
| SRBD1.6 | FWTAYI (SEQ ID | 3.91 × 10⁵ | 1.33 × 10⁻⁴ | 0.339 |
| | NO: 29) | | | |
| SRBD1.7 | FFNSYI (SEQ ID | 1.39 × 10⁶ | 1.72 × 10⁻⁴ | 0.123 |
| | NO: 30) | | | |
| SRBD1.8 | FHNIYL (SEQ ID | 6.47 × 10⁵ | 7.23 × 10⁻⁵ | 0.118 |
| | NO: 31) | | | |
| SRBD1.9 | FHNLYV (SEQ ID | 7.05 × 10⁵ | 5.00 × 10⁻⁵ | 0.071 |
| | NO: 32) | | | |
| SRBD1.10 | FHRAYI (SEQ ID | 1.38 × 10⁶ | 2.75 × 10⁻⁴ | 0.199 |
| | NO: 33) | | | |
| SRBD1.11 | FHRIYI (SEQ ID | 9.03 × 10⁵ | 5.23 × 10⁻⁴ | 0.560 |
| | NO: 34) | | | |
| SRBD1.12 | FWASYI (SEQ ID | 9.49 × 10⁵ | 2.92 × 10⁻⁴ | 0.307 |
| | NO: 35) | | | |
| SRBD1.13 | FYASYI (SEQ ID | 7.86 × 10⁵ | 3.34 × 10⁻⁴ | 0.425 |
| | NO: 36) | | | |
| SRBD1.14 | LANNYV (SEQ ID | 5.21 × 10⁵ | 3.62 × 10⁻⁴ | 0.693 |
| | NO: 37) | | | |
| SRBD1.15 | FHNSYI (SEQ ID | 1.07 × 106 | 1.14 × 10⁻⁴ | 0.107 |
| | NO: 38) | | | |
| SRBD1.16 | FHRTYI (SEQ ID | 1.12 × 106 | 2.47 × 10⁻⁴ | 0.221 |
| | NO: 39) | | | |
| SRBD1.17 | LNNIYI (SEQ ID | 5.84 × 105 | 1.07 × 10⁻⁴ | 0.183 |
| | NO: 40) | | | |
| SRBD1.18 | VHWVYF (SEQ | 1.26 × 106 | 3.54 × 10⁻⁴ | 0.273 |
| | ID NO: 41) | | | |
| SRBD1.19 | LHNQYL (SEQ ID | 7.02 × 105 | 1.27 × 10⁻⁴ | 0.181 |
| | NO: 42) | | | |

TABLE 6

| Fab | Kₒₙ (M⁻¹ s⁻¹) | K_{off} (s⁻¹) | K_{D} (nM) | χ² (RU²) |
|---|---|---|---|---|
| sRBD1.15 | 1.0 × 10⁶ | 1.1 × 10⁻⁴ | 0.1 | 3.36 |

### Example 4—Detection of SARS-CoV-2 with GA1-FAB′ Complementation Assay

A GA1-FAB^{LRT} protein complementation assay was developed that was successfully applied for the detection of Ebola and Zika virus proteins 4. The system involves the use of the well-established proximity-driven activity reconstitution of a TEM1 β-lactamase (BL) split enzyme system⁵. In this assay, the two separate fragments of the BL (BLF1, residues 26-196, and BLF2, residues 198-220) are attached through a linker to engineered protein G-A1⁶. Protein complementation compositions, materials, and methods are disclosed in, for example, U.S. Pat. No. 10,759,834, incorporated herein by reference in its entirety.

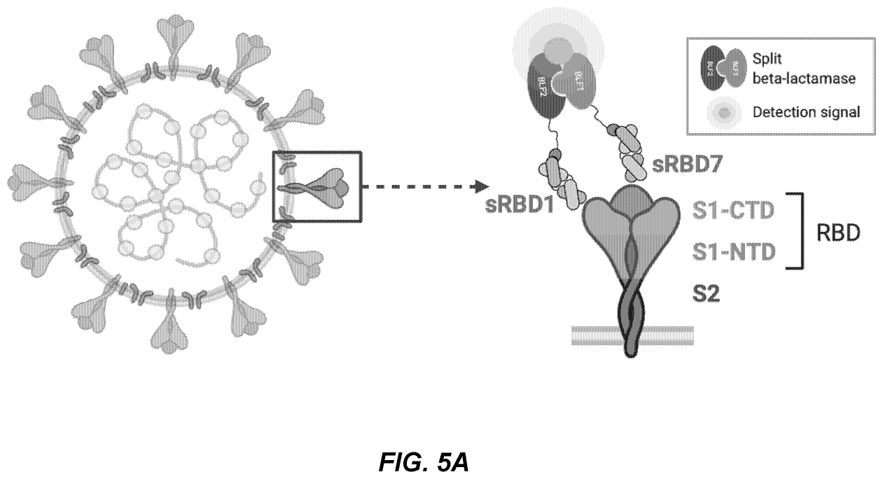

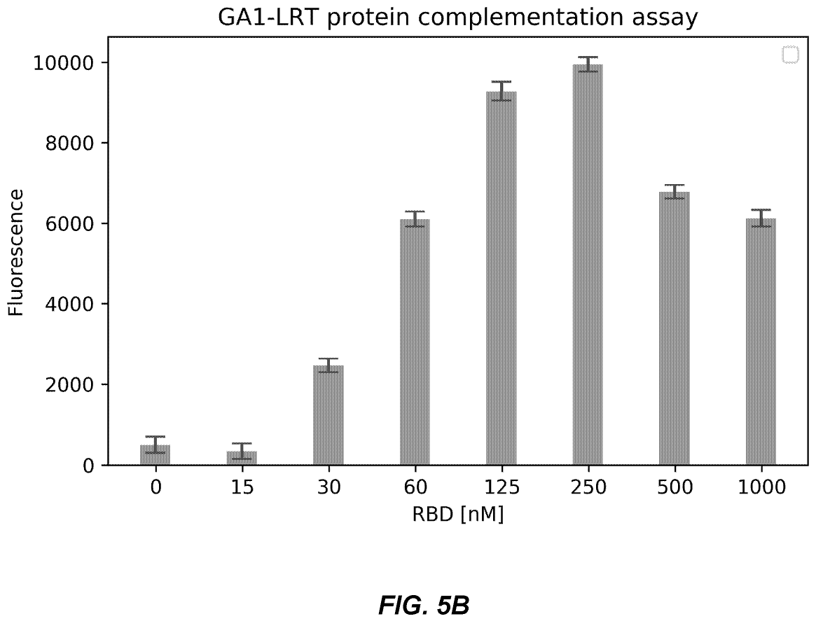

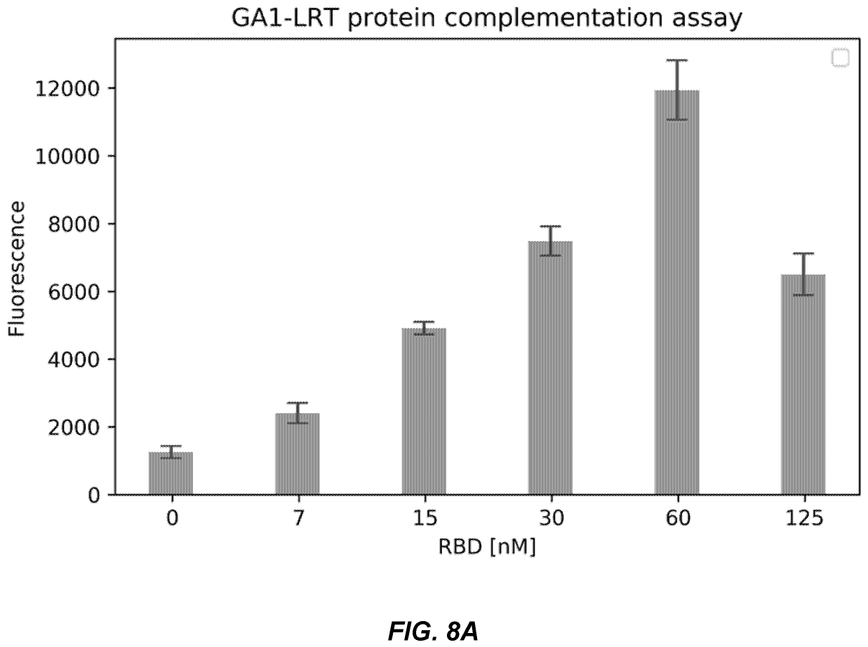

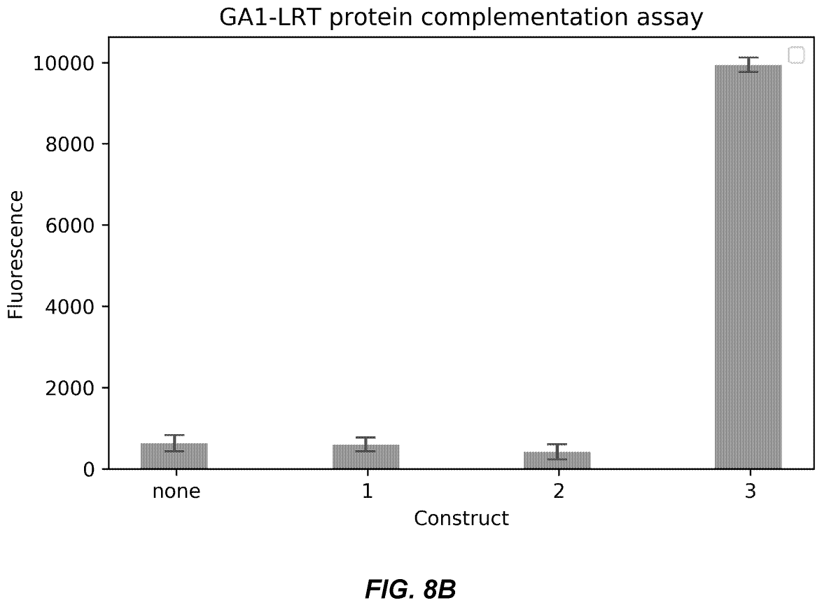

Using the ultra-high affinity of Fab^{LRT} scaffold⁴ to protein G-A1, the inventors were able to generate a pair of sRBD1-BLF2 and sRBD7-BLF1 by premixing them separately to a final concentration of 250 nM. Such a non-wash assay requires simultaneous binding of two Fabs to the target protein for successful detection, as shown in FIG. 5A. Without knowledge of the sRBD1 and sRBD7 epitopes' positions, a Gly-Ser linker of 15 residues was used between protein G-BLF constructs. Titration of RBD resulted in a detectable fluorescent signal at 30 nM, which increased linearly over the range from 30 to 125 nM with a distinct maximum at 250 nM (FIG. 5B). A reduction of the signal was observed at RBD excess, presumably caused by a breakdown of the stoichiometry at high antigen concentration due to the hook effect. To establish how the assay is influenced by the affinity levels of the Fab, the sRBD1 Fab (KD˜3 nM) was replaced with the sRBD1.5 Fab (KD˜42 pM) coupled to BLF2, while keeping sRBD7 attached to BLF1. This resulted in about ˜4-fold improvement in detection (FIG. 8A). Control experiments with premixing the same Fab (sRBD1 or sRBD7) with both BLF constructs did not show a detectable signal in a presence of 250 nM RBD (FIG. 8B).

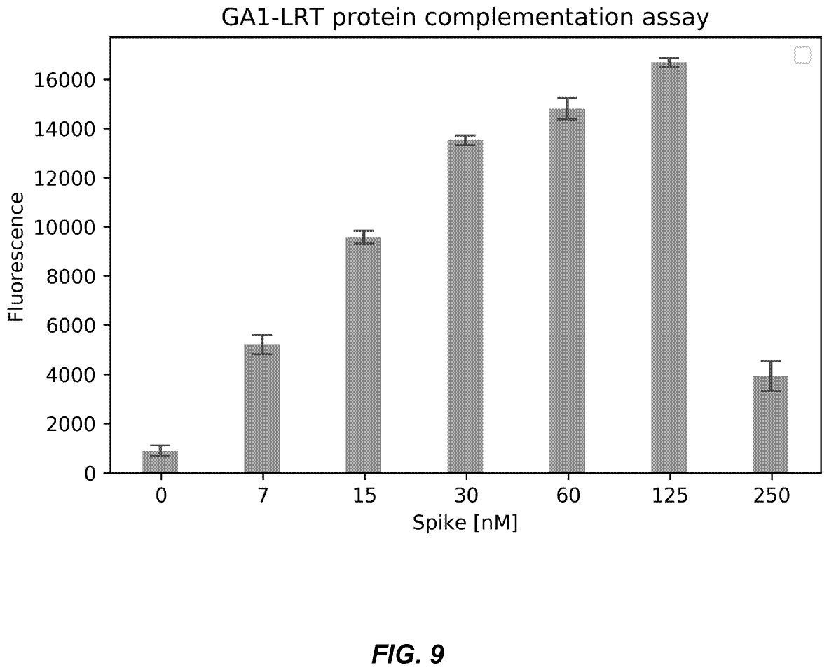

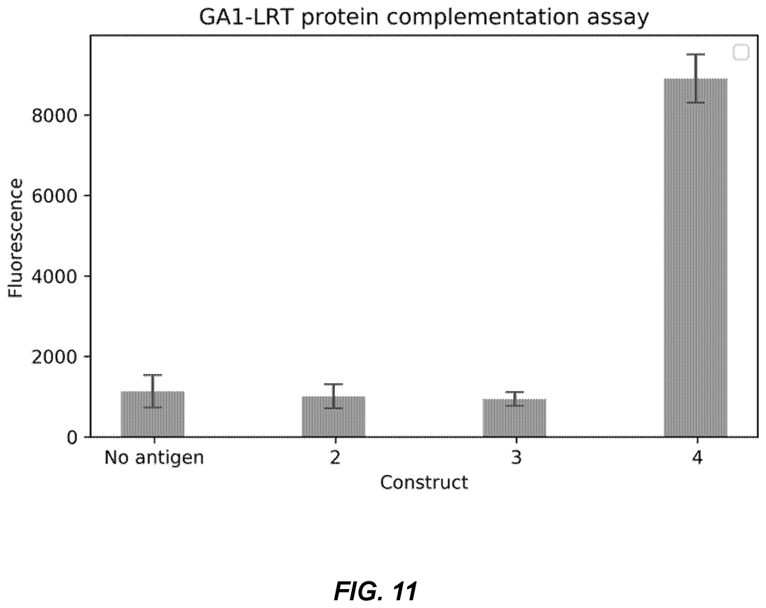

Next, the inventors tested whether this system could be used for detection of full trimeric spike (S) protein, since the sRBD Fab epitopes might be not accessible in a context of a full-length spike protein. The spike protein was readily detected with a higher sensitivity with a detectable concentration starting at 7 nM with a distinct maximum at 125 nM (FIG. 9). A control experiment, where each Fab was premixed with both components (i.e. sRBD1-BLF1, sRBD1-BLF2) showed that measurable signal was not produced in this format (FIG. 11). Thus, as was the case for the RBD domain alone, triggering a signal required the Fabs to bind to two independent epitopes on the same trimeric S-protein configuration.

### Example 6—Post Lyophilization Stability of pGA1-FAB′ Complementation Assay

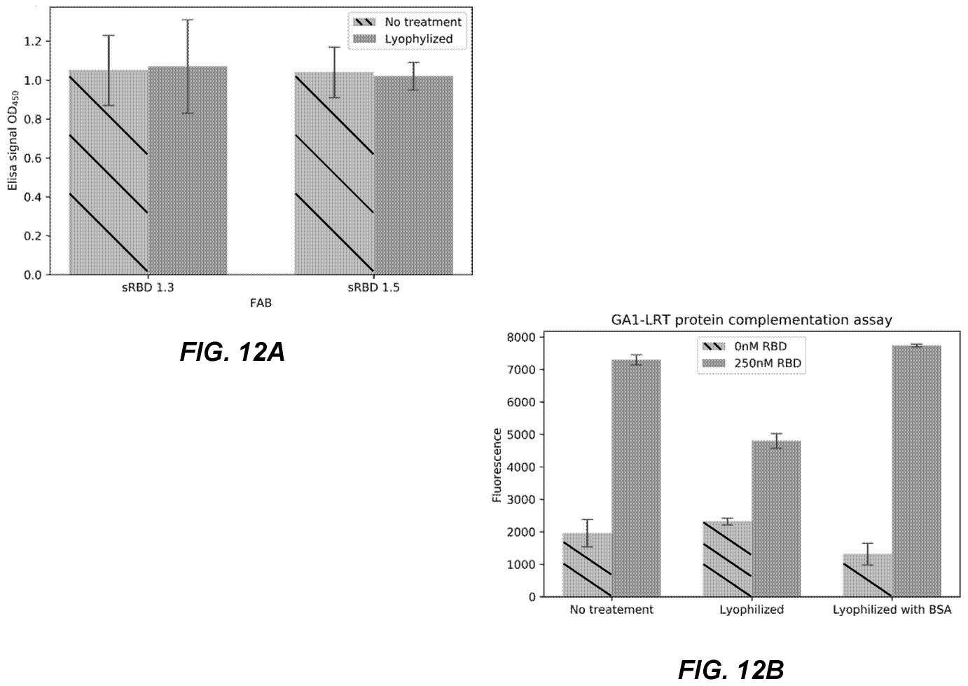

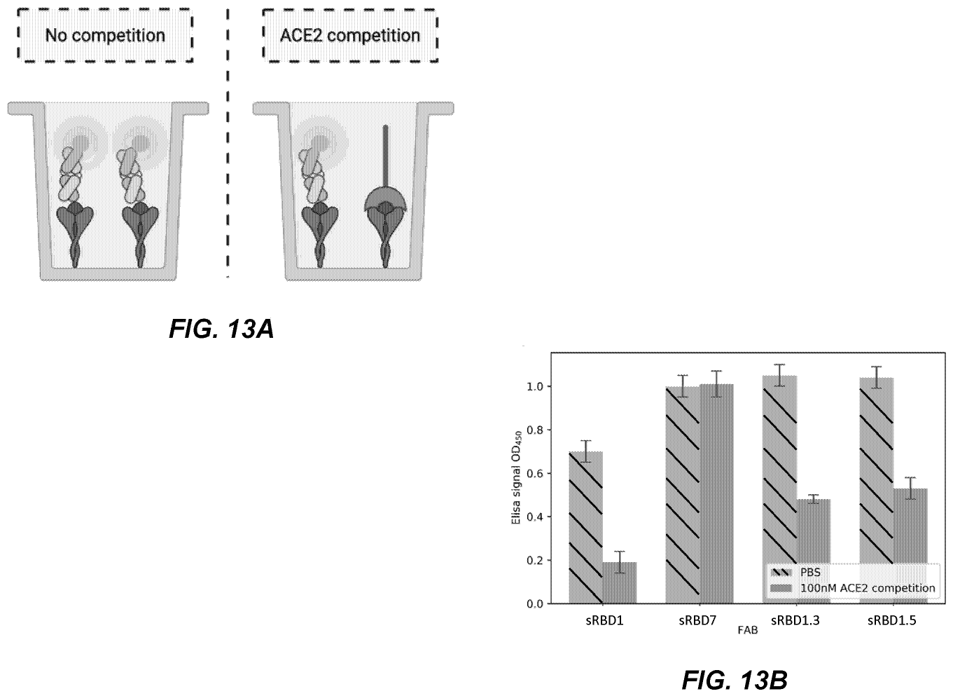

Single Fabs, sRBD1-BLF2 and sRBD7-BLF1 at 500 nM, were lyophilized overnight. Upon reconstitution, no aggregation was observed and the Fabs' binding properties were unchanged (FIG. 12A). Moreover, the pGA1-FAB^{LRT} complementation assay performed equally well as the control (not lyophilized) when the sample was supplemented with a protein carrier (BSA) as part of the freeze-drying procedure. Lack of BSA supplementation led to 40% signal reduction FIG. 12B.

### Example 7—Example Methods

Protein Cloning, Expression and Purification

The RBD protein was produced in mammalian cells (gifted by Wilson and Hubbell labs from the University of Chicago). Proteins for the split enzyme proximity assay were cloned into pHFT2 vector18 with the strategy described previously.3 Selected Fabs were cloned from phage into Sph1 sites of pSFV4 expression vector using an Infusion HD cloning kit (Takara Bio) according to the protocol. All selected Fab CDR sequences are provided in Table 51. The Fab LRT scaffold was grafted into Fab light chain at aa positions 123-127 using quick change site-directed mutagenesis.

Fabs and Fab-GA1 fusions were expressed in the periplasm of E. coli BL21 cells for 4 hours at 37° C. post induction with 1 mM IPTG at OD₆₀₀=0.8-1. The cells were harvested by sonication in Protein G-wash buffer (50 mM Phosphate buffer, 500 mM NaCl, pH 7.4). After centrifugation the supernatant was applied on the protein G-F affinity column created in the lab using SulfoLink Coupling Resin (Thermo Scientific). Proteins were eluted from the column with 0.1 M glycine, pH 2.6, and neutralized with 1 M Tris-HCl, pH 8.5. Fabs and Fab-GA1 fusions were dialyzed overnight into HBS.

For the proximity assay, pGA1-BLF fusions were expressed and purified as described previously. Briefly, proteins were expressed in BL21 (DE3) overnight in 20° C. post induction with 1 mM IPTG at ° Da)=0.6. Cells were sonicated in buffer A containing 50 mM Tris-HCl, pH 8.0, 150 mM NaCl and 10% glycerol. The insoluble His-tagged pGA1-BLF fusions were extracted from the cell pellets by 6 M Gua-HCl in buffer A with 0.3 mM TCEP and purified on TALON resin (Takara Bio) using a denaturation-condition protocol and on-column renaturations reached by six washes of the column with serially diluted 6 M Gua-HCl, followed by the final wash in buffer A alone. Proteins were eluted from the column with 100 mM imidazole in buffer A. Fusion proteins were stored on ice and never frozen.

Phage Display Selection Protocol

To obtain high affinity binders, five rounds of selection were performed using phage display selection protocol. RBD protein was biotinylated via glycoproteins with EZ-Link Hydrazide Biotin (Thermo Scientific) as recommended in order to immobilize onto streptavidin-coated paramagnetic beads (Promega and Dynabeads M0270, Invitrogen). In the first round of selection, 500 nM of RBD was immobilized on 200 μl SA magnetic beads (Promega) and was incubated with 1 mL phage library (10¹¹ cfu) for 1 hour at room temperature with gentle shaking. The beads were washed three times to remove nonspecific phage and added to log phase E. coli XL-1 blue cells (Stratagene) and incubated for 20 min at room temperature. Then, media containing 100 m/mL ampicillin and 10⁹ p.f.u./mL of M13K07 helper phage (NEB) was added for overnight phage amplification at 37° C. For all subsequent rounds, the amplified phage was precipitated in 20% PEG/2.5 M NaCl for 20 min on ice. Before each round, the phage pool was negatively selected against empty paramagnetic beads for 30 min with shaking to eliminate nonspecific binders. The final concentration of antigen was dropped systematically from 500 to 1 nM from the first to the fifth round (2nd round: 200 nM, 3rd round: 50 nM, 4th round 10 nM and 5th round 1 nM). After phage binding, the beads were subjected to five washing rounds. Last two washes steps were 20 and 25 min respectively. This additional selection pressure was supposed to remove all low affinity binders. The bound phages were eluted using 0.1 M glycine, pH 2.6 and neutralized with TRIS-HCl, pH 8. Then, the phage eluate was used for E. coli infection and phage amplification as described above. Additional selection to generate non-overlapping epitope with sRBD1 was performed. Selection protocol was as described above with addition of 1 μM of sRBD1 in every step to ensure binding to a different epitope. The final concentration of antigen was dropped gradually from 200 to 1 nM from the first to the fifth round (2nd round: 50 nM, 3rd round: 20 nM, 4th round 10 nM and 5th round 1 nM). After round 4th and 5th phages were plated on ampicillin plates and 96 single colonies were picked for single-point phage ELISA assays. The promising clones demonstrating high ELISA signal and low non-specific binding were sequenced and reformatted into a pSFV4 expressing vector as described in Protein expression and purification.

Enzyme-Linked Immunoabsorbent Assays (ELISA)

The ELISA protocol was described previously. Briefly, 50 nM of RBD was directly immobilized on high binding experimental wells (Greiner Bio) and BSA was immobilized in control wells, followed by extensive blocking with BSA. After 15 min incubation with phage, the wells were extensively washed three times and incubated with Protein L-HRP (Thermo Scientific, 1:5000 dilution in HBST) for 20 min. The plates were again washed and developed with TMB substrate (Thermo Scientific) and quenched with 10% H₃PO₄, followed by the absorbance at A₄₅₀ determination.

Affinity Maturation of RBD1

Phage libraries of RBD1 for affinity maturation were created using the strategy previously published. To that end, a stop codon was introduced in CDR-H1 with quick-change mutagenesis. Two phage libraries with “hard” and “tailored” randomization strategies were created with the phosphorylated oligos. ssDNA containing stop codon introduced in the middle of CDR-H1 was isolated from phage (using QIAprep Spin M13 Kit, Qiagen) and used in a Kunkel mutagenesis protocol. The Kunkel reaction was purified (with Wizard SV Gel and PCR Clean-Up System, Promega), electroporated into TG1 cells (Lucigen) and after 1-hour recovery in 37° C. with shaking, 40 mL of 2xYT media (supplemented with 100 m/mL ampicillin and helper phage) was added to initiate phage production. The next day, the libraries were precipitated as described above. Three rounds of biopanning were performed with different target concentration (1st round: 10 nM, 2nd round: 1 nM, 3rd round: 20 pM). To ensure the improvement in dissociation constant, selection process included 4 consecutive 30 min washing steps. Affinity improvement of the selected clones was tested by SPR.

Surface Plasmon Resonance Analysis

All Surface plasmon resonance (SPR) analyses were performed on a MASS-1 (Bruker). All targets were immobilized via a 6× His-tag to a Ni-NTA sensor chip. Fabs in twofold dilutions were run as analytes at 30111/min flow rate at 20° C. Sensograms were corrected through double referencing and 1:1 binding model fit was done using Sierra Analyzer (Bruker). For epitope binning experiments, after injection of a saturating concentration of the sRBD1 Fab, an equal molar mixture of sRBD7 fab was injected and increase in Response Unit (RU) was observed.

pGA1-FAB^{LRT} Protein Complementation Assay

Viral detection protocol using non-wash pGA1-FAB^{LRT} protein complementation assay was previously described. Briefly, 250 nM of each pGA1-BLF fusion, premixed with RBD1 or RBD7 Fabs, were combined in a black FluoroNunc 96-well plate (Nunc). Different concentrations of viral proteins and 2 μM fluorogenic BL substrate (Fluorocillin Green 495/525, Life Technologies) was added to the final volume of 100 μl. Fluorescent signal was monitored at room temperature using Safire2 Tecan Plate Reader (483 nm excitation, 525 nm emission). The results were reproduced at least three times. Results were normalized by subtraction of a substrate background fluorescence.

Plaque Reduction Neutralization Assay

Vero E6 cells (ATCC) were infected under biosafety level 3 conditions with SARS-CoV-2 (nCoV/Washington/1/2020, kindly provided by the National Biocontainment Laboratory, Galveston, TX). The neutralization assay was performed as previously described with some modifications. Briefly, the Fabs were serially diluted 4-fold and mixed with 400 PFU of SARS-CoV-2 for one hour at 37° C., then used to infect Vero E6 cells for three days. Cells were fixed with 3.7% formalin and stained with 0.25% crystal violent. Crystal violet-stained cells were then quantified by absorbance at (595 nm) with a Tecan m200 microplate reader. The 50% neutralization titer was then calculated using GraphPad Prism.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Miller K R, Koide A, Leung B, Fitzsimmons J, Yoder B, Yuan H, Jay M, Sidhu S S, Koide S, Collins E J. 2012. T cell receptor-like recognition of tumor in vivo by synthetic antibody fragment. PLoS One. 7(8):e43746.
2. Paduch M, Koide A, Uysal S, Rizk S S, Koide S, Kossiakoff A A. 2013. Generating conformation-specific synthetic antibodies to trap proteins in selected functional states. Methods. 60(1):3-14.
3. Fellouse F A, Esaki K, Birtalan S, Raptis D, Cancasci V J, Koide A, Jhurani P, Vasser M, Wiesmann C, Kossiakoff A A et al. 2007. High-throughput generation of synthetic antibodies from highly functional minimalist phage-displayed libraries. J Mol Biol. 373(4):924-940.
4. Slezak T, Bailey L J, Jaskolowski M, Nahotko D A, Filippova E V, Davydova E K, Kossiakoff A A. 2020. An engineered ultra-high affinity fab-protein g pair enables a modular antibody platform with multifunctional capability. Protein Sci. 29(1):141-156.
5. Galarneau A, Primeau M, Trudeau L E, Michnick S W. 2002. Beta-lactamase protein fragment complementation assays as in vivo and in vitro sensors of protein protein interactions. Nat Biotechnol. 20(6):619-622.
6. Bailey L J, Sheehy K M, Hoey R J, Schaefer Z P, Ura M, Kossiakoff A A. 2014. Applications for an engineered protein-g variant with a ph controllable affinity to antibody fragments. J Immunol Methods. 415:24-30.

1. A polypeptide that specifically binds to a SARS-CoV-2 spike protein, comprising:
(a) a heavy chain variable region (VH) comprising:
(i) a CDR-H1 comprising a sequence having at least 80% identity to SEQ ID NO:1, SEQ ID NO:2, SEQ ID NO:3, SEQ ID NO:4, or SEQ ID NO:5;
(ii) a CDR-H2 comprising a sequence having at least 80% identity to SEQ ID NO:6, SEQ ID NO:113, or SEQ ID NO:7; and
(iii) a CDR-H3 comprising a sequence having at least 80% identity to SEQ ID NO:8, SEQ ID NO:9, SEQ ID NO:10, SEQ ID NO:11, SEQ ID NO:12, SEQ ID NO:13, or SEQ ID NO:14; and
(b) a light chain variable region (VL) comprising:
(i) a CDR-L1 comprising a sequence having at least 80% identity to SEQ ID NO:15;
(ii) a CDR-L2 comprising a sequence having at least 80% identity to SEQ ID NO:16; and
(iii) a CDR-L3 comprising a sequence having at least 80% identity to SEQ ID NO:17, SEQ ID NO:18, SEQ ID NO:19, SEQ ID NO:20, SEQ ID NO:21, SEQ ID NO:22, or SEQ ID NO:23.

2. (canceled)

3. The polypeptide of claim 1, wherein the CDR-H1 comprises SEQ ID NO:1, SEQ ID NO:2, SEQ ID NO:3, SEQ ID NO:4, or SEQ ID NO:5.

4.-8. (canceled)

9. The polypeptide of claim 1, wherein the CDR-H2 comprises SEQ ID NO:6, SEQ ID NO:113, or SEQ ID NO:7.

10.-12. (canceled)

13. The polypeptide of claim 1, wherein the CDR-H3 comprises SEQ ID NO:8, SEQ ID NO:9, SEQ ID NO:10, SEQ ID NO:11, SEQ ID NO:12, SEQ ID NO:13, or SEQ ID NO:14.

14.-20. (canceled)

21. The polypeptide of claim 1, wherein the CDR-L1 comprises SEQ ID NO:15.

22. The polypeptide of claim 1, wherein the CDR-L2 comprises SEQ ID NO:16.

23. The polypeptide of claim 1, wherein the CDR-L3 comprises SEQ ID NO:17, SEQ ID NO:18, SEQ ID NO:19, SEQ ID NO:20, SEQ ID NO:21, SEQ ID NO:22, or SEQ ID NO:23.

24.-30. (canceled)

31. The polypeptide of claim 1, wherein the VH comprises a sequence having at least 80% identity to SEQ ID NO:44, SEQ ID NO:46, SEQ ID NO:48, SEQ ID NO:50, SEQ ID NO:52, SEQ ID NO:54, or SEQ ID NO:56.

32. The polypeptide of claim 31, wherein the VH comprises SEQ ID NO:44, SEQ ID NO:46, SEQ ID NO:48, SEQ ID NO:50, SEQ ID NO:52, SEQ ID NO:54, or SEQ ID NO:56.

33.-39. (canceled)

40. The polypeptide of claim 31, wherein the VL comprises a sequence having at least 80% identity to SEQ ID NO:45, SEQ ID NO:47, SEQ ID NO:49, SEQ ID NO:51, SEQ ID NO:53, SEQ ID NO:55, or SEQ ID NO:57.

41. The polypeptide of claim 40, wherein the VL comprises SEQ ID NO:45, SEQ ID NO:47, SEQ ID NO:49, SEQ ID NO:51, SEQ ID NO:53, SEQ ID NO:55, or SEQ ID NO:57.

42.-51. (canceled)

52. The polypeptide of claim 1, wherein the polypeptide is an antibody or antigen-binding fragment thereof.

53. The polypeptide of claim 52, wherein the polypeptide is a human antibody, humanized antibody, recombinant antibody, chimeric antibody, an antibody derivative, a veneered antibody, a diabody, a monoclonal antibody, or a polyclonal antibody.

54.-55. (canceled)

56. The polypeptide of claim 1, wherein the polypeptide is a Fab.

57.-79. (canceled)

80. A nucleic acid encoding for the polypeptide of claim 1.

81.-90. (canceled)

91. A method for treating a subject for a SARS-CoV-2 infection, the method comprising administering to the subject an effective amount of a composition comprising the polypeptide of claim 1.

92. A method for detecting a SARS-CoV-2 spike protein in a sample, the method comprising:
(a) providing to the sample
(i) a first polypeptide operatively linked to a first component of a detection pair; and
(ii) a second polypeptide operatively linked to a second component of the detection pair; and
(b) detecting the detection pair,
wherein the first polypeptide specifically binds to a first region of the SARS-CoV-2 spike protein and the second polypeptide specifically binds to a second region of the SARS-CoV-2 spike protein.

93. The method of claim 92, wherein the detection pair comprises an enzyme and detecting the detection pair comprises detecting enzymatic activity of the enzyme.

94.-96. (canceled)

97. The method of claim 92, wherein the detection pair comprises a donor fluorophore and an acceptor fluorophore.

98.-120. (canceled)

121. A method for treating a subject for a SARS-CoV-2 infection, the method comprising administering to the subject a therapeutically effective amount of a polypeptide comprising (a) a sequence having at least 80% identity to one of SEQ ID NOs:77-83; and (b) a sequence having at least 80% identity to one of SEQ ID NOs:84-109.

122.-129. (canceled)


SEQUENCE LISTING (121 sequences)

SEQ ID NO: 1 - protein, 6 aa, Artificial Sequence
  Synthetic Polypeptide
FSSSYI

SEQ ID NO: 2 - protein, 6 aa, Artificial Sequence
  Synthetic Polypeptide
VYSSYI

SEQ ID NO: 3 - protein, 6 aa, Artificial Sequence
  Synthetic Polypeptide
IYSSYI

SEQ ID NO: 4 - protein, 6 aa, Artificial Sequence
  Artificial Polypeptide
LYSSYI

SEQ ID NO: 5 - protein, 6 aa, Artificial Sequence
  Artificial Polypeptide
VSSSYI

SEQ ID NO: 6 - protein, 10 aa, Artificial Sequence
  Artificial Polypeptide
SISSYSGYTY

SEQ ID NO: 7 - protein, 10 aa, Artificial Sequence
  Artificial Polypeptide
YISPYYGSTY

SEQ ID NO: 8 - protein, 8 aa, Artificial Sequence
  Artificial Polypeptide
SYQGQEAL

SEQ ID NO: 9 - protein, 6 aa, Artificial Sequence
  Artificial Polypeptide
YAGHAM

SEQ ID NO: 10 - protein, 6 aa, Artificial Sequence
  Artificial Polypeptide
YGDDAM

SEQ ID NO: 11 - protein, 6 aa, Artificial Sequence
  Artificial Polypeptide
YHSWAF

SEQ ID NO: 12 - protein, 7 aa, Artificial Sequence
  Artificial Polypeptide
YSYPGGM

SEQ ID NO: 13 - protein, 7 aa, Artificial Sequence
  Artificial Polypeptide
YAEGMGM

SEQ ID NO: 14 - protein, 7 aa, Artificial Sequence
  Artificial Polypeptide
YWSYGGM

SEQ ID NO: 15 - protein, 5 aa, Artificial Sequence
  Artificial Polypeptide
SVSSA

SEQ ID NO: 16 - protein, 7 aa, Artificial Sequence
  Artificial Polypeptide
SASSLYS

SEQ ID NO: 17 - protein, 7 aa, Artificial Sequence
  Artificial Polypeptide
YLYFSLV

SEQ ID NO: 18 - protein, 7 aa, Artificial Sequence
  Artificial Polypeptide
SNYSYPI

SEQ ID NO: 19 - protein, 7 aa, Artificial Sequence
  Artificial Polypeptide
ASWGSPI

SEQ ID NO: 20 - protein, 7 aa, Artificial Sequence
  Artificial Polypeptide
SYYGSLI

SEQ ID NO: 21 - protein, 7 aa, Artificial Sequence
  Artificial Polypeptide
SYYGNLI

SEQ ID NO: 22 - protein, 7 aa, Artificial Sequence
  Artificial Polypeptide
HAWGYLI

SEQ ID NO: 23 - protein, 7 aa, Artificial Sequence
  Artificial Polypeptide
YYYGALI

SEQ ID NO: 24 - protein, 6 aa, Artificial Sequence
  Artificial Polypeptide
FGGSYI

SEQ ID NO: 25 - protein, 6 aa, Artificial Sequence
  Artificial Polypeptide
FHGIYL

SEQ ID NO: 26 - protein, 6 aa, Artificial Sequence
  Artificial Polypeptide
FHSTYI

SEQ ID NO: 27 - protein, 6 aa, Artificial Sequence
  Artificial Polypeptide
FWLGYI

SEQ ID NO: 28 - protein, 6 aa, Artificial Sequence
  Artificial Polypeptide
FWNSYI

SEQ ID NO: 29 - protein, 6 aa, Artificial Sequence
  Artificial Polypeptide
FWTAYI

SEQ ID NO: 30 - protein, 6 aa, Artificial Sequence
  Artificial Polypeptide
FFNSYI

SEQ ID NO: 31 - protein, 6 aa, Artificial Sequence
  Artificial Polypeptide
FHNIYL

SEQ ID NO: 32 - protein, 6 aa, Artificial Sequence
  Artificial Polypeptide
FHNLYV

SEQ ID NO: 33 - protein, 6 aa, Artificial Sequence
  Artificial Polypeptide
FHRAYI

SEQ ID NO: 34 - protein, 6 aa, Artificial Sequence
  Artificial Polypeptide
FHRIYI

SEQ ID NO: 35 - protein, 6 aa, Artificial Sequence
  Artificial Polypeptide
FWASYI

SEQ ID NO: 36 - protein, 6 aa, Artificial Sequence
  Artificial Polypeptide
FYASYI

SEQ ID NO: 37 - protein, 6 aa, Artificial Sequence
  Artificial Polypeptide
LANNYV

SEQ ID NO: 38 - protein, 6 aa, Artificial Sequence
  Artificial Polypeptide
FHNSYI

SEQ ID NO: 39 - protein, 6 aa, Artificial Sequence
  Artificial Polypeptide
FHRTYI

SEQ ID NO: 40 - protein, 6 aa, Artificial Sequence
  Artificial Polypeptide
LNNIYI

SEQ ID NO: 41 - protein, 6 aa, Artificial Sequence
  Artificial Polypeptide
VHWVYF

SEQ ID NO: 42 - protein, 6 aa, Artificial Sequence
  Artificial Polypeptide
LHNQYL

SEQ ID NO: 43 - protein, 6 aa, Artificial Sequence
  Artificial Polypeptide
  MISC_FEATURE (1)..(1): X is Phe or Leu or Val
  MISC_FEATURE (2)..(2): X is His or Trp or Ala or Phe or Gly or Asn or Tyr
  MISC_FEATURE (3)..(3): X is Asn or Arg or Ala or Gly or Leu or Ser or Thr or Trp
  MISC_FEATURE (4)..(4): X is Ser or Ile or Ala or Thr or Gly or Leu or Asn or Gln or Val
  MISC_FEATURE (6)..(6): X is Ile or Leu or Val or Phe
XXXXYX

SEQ ID NO: 44 - protein, 122 aa, Artificial Sequence
  Artificial Polypeptide
EISEVQLVESGGGLVQPGGSLRLSCAASGFNFSSSYIHWVRQAPGKGLEWVASISSYSGYTYYADSVKGRFTISADTSKNTAYLQMNSLRAEDTAVYYCARSYQGQEALDYWGQGTLVTVSS

SEQ ID NO: 45 - protein, 109 aa, Artificial Sequence
  Artificial Polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQYLYFSLVTFGQGTKVEIK

SEQ ID NO: 46 - protein, 120 aa, Artificial Sequence
  Artificial Polypeptide
EISEVQLVESGGGLVQPGGSLRLSCAASGFNVYSSYIHWVRQAPGKGLEWVAYISSYYGSTYYADSVKGRFTISADTSKNTAYLQMNSLRAEDTAVYYCARYAGHAMDYWGQGTLVTVSS

SEQ ID NO: 47 - protein, 109 aa, Artificial Sequence
  Artificial Polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQSNYSYPITFGQGTKVEIK

SEQ ID NO: 48 - protein, 120 aa, Artificial Sequence
  Artificial Polypeptide
EISEVQLVESGGGLVQPGGSLRLSCAASGFNIYSSYIHWVRQAPGKGLEWVAYISSYYGSTYYADSVKGRFTISADTSKNTAYLQMNSLRAEDTAVYYCARYGDDAMDYWGQGTLVTVSS

SEQ ID NO: 49 - protein, 109 aa, Artificial Sequence
  Artificial Polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQASWGSPITFGQGTKVEIK

SEQ ID NO: 50 - protein, 120 aa, Artificial Sequence
  Artificial Polypeptide
EISEVQLVESGGGLVQPGGSLRLSCAASGFNLYSSYIHWVRQAPGKGLEWVAYISSYYGSTYYADSVKGRFTISADTSKNTAYLQMNSLRAEDTAVYYCARYHSWAFDYWGQGTLVTVSS

SEQ ID NO: 51 - protein, 109 aa, Artificial Sequence
  Artificial Polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQSYYGSLITFGQGTKVEIK

SEQ ID NO: 52 - protein, 121 aa, Artificial Sequence
  Artificial Polypeptide
EISEVQLVESGGGLVQPGGSLRLSCAASGFNVYSSYIHWVRQAPGKGLEWVAYISSYYGSTYYADSVKGRFTISADTSKNTAYLQMNSLRAEDTAVYYCARYSYPGGMDYWGQGTLVTVSS

SEQ ID NO: 53 - protein, 109 aa, Artificial Sequence
  Artificial Polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQSYYGNLITFGQGTKVEIK

SEQ ID NO: 54 - protein, 121 aa, Artificial Sequence
  Artificial Polypeptide
EISEVQLVESGGGLVQPGGSLRLSCAASGFNVSSSYIHWVRQAPGKGLEWVAYISSYYGSTYYADSVKGRFTISADTSKNTAYLQMNSLRAEDTAVYYCARYAEGMGMDYWGQGTLVTVSS

SEQ ID NO: 55 - protein, 109 aa, Artificial Sequence
  Artificial Polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQHAWGYLITFGQGTKVEIK

SEQ ID NO: 56 - protein, 121 aa, Artificial Sequence
  Artificial Polypeptide
EISEVQLVESGGGLVQPGGSLRLSCAASGFNVYSSYIHWVRQAPGKGLEWVAYISPYYGSTYYADSVKGRFTISADTSKNTAYLQMNSLRAEDTAVYYCARYWSYGGMDYWGQGTLVTVSS

SEQ ID NO: 57 - protein, 109 aa, Artificial Sequence
  Artificial Polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQYYYGALITFGQGTKVEIK

SEQ ID NO: 58 - protein, 122 aa, Artificial Sequence
  Artificial Polypeptide
EISEVQLVESGGGLVQPGGSLRLSCAASGFNFGGSYIHWVRQAPGKGLEWVASISSYSGYTYYADSVKGRFTISADTSKNTAYLQMNSLRAEDTAVYYCARSYQGQEALDYWGQGTLVTVSS

SEQ ID NO: 59 - protein, 122 aa, Artificial Sequence
  Artificial Polypeptide
EISEVQLVESGGGLVQPGGSLRLSCAASGFNFHGIYLHWVRQAPGKGLEWVASISSYSGYTYYADSVKGRFTISADTSKNTAYLQMNSLRAEDTAVYYCARSYQGQEALDYWGQGTLVTVSS

SEQ ID NO: 60 - protein, 122 aa, Artificial Sequence
  Artificial Polypeptide
EISEVQLVESGGGLVQPGGSLRLSCAASGFNFHSTYIHWVRQAPGKGLEWVASISSYSGYTYYADSVKGRFTISADTSKNTAYLQMNSLRAEDTAVYYCARSYQGQEALDYWGQGTLVTVSS

SEQ ID NO: 61 - protein, 122 aa, Artificial Sequence
  Artificial Polypeptide
EISEVQLVESGGGLVQPGGSLRLSCAASGFNFWLGYIHWVRQAPGKGLEWVASISSYSGYTYYADSVKGRFTISADTSKNTAYLQMNSLRAEDTAVYYCARSYQGQEALDYWGQGTLVTVSS

SEQ ID NO: 62 - protein, 122 aa, Artificial Sequence
  Artificial Polypeptide
EISEVQLVESGGGLVQPGGSLRLSCAASGFNFWNSYIHWVRQAPGKGLEWVASISSYSGYTYYADSVKGRFTISADTSKNTAYLQMNSLRAEDTAVYYCARSYQGQEALDYWGQGTLVTVSS

SEQ ID NO: 63 - protein, 122 aa, Artificial Sequence
  Artificial Polypeptide
EISEVQLVESGGGLVQPGGSLRLSCAASGFNFWTAYIHWVRQAPGKGLEWVASISSYSGYTYYADSVKGRFTISADTSKNTAYLQMNSLRAEDTAVYYCARSYQGQEALDYWGQGTLVTVSS

SEQ ID NO: 64 - protein, 122 aa, Artificial Sequence
  Artificial Polypeptide
EISEVQLVESGGGLVQPGGSLRLSCAASGFNFFNSYIHWVRQAPGKGLEWVASISSYSGYTYYADSVKGRFTISADTSKNTAYLQMNSLRAEDTAVYYCARSYQGQEALDYWGQGTLVTVSS

SEQ ID NO: 65 - protein, 122 aa, Artificial Sequence
  Artificial Polypeptide
EISEVQLVESGGGLVQPGGSLRLSCAASGFNFHNIYLHWVRQAPGKGLEWVASISSYSGYTYYADSVKGRFTISADTSKNTAYLQMNSLRAEDTAVYYCARSYQGQEALDYWGQGTLVTVSS

SEQ ID NO: 66 - protein, 122 aa, Artificial Sequence
  Artificial Polypeptide
EISEVQLVESGGGLVQPGGSLRLSCAASGFNFHNLYVHWVRQAPGKGLEWVASISSYSGYTYYADSVKGRFTISADTSKNTAYLQMNSLRAEDTAVYYCARSYQGQEALDYWGQGTLVTVSS

SEQ ID NO: 67 - protein, 122 aa, Artificial Sequence
  Artificial Polypeptide
EISEVQLVESGGGLVQPGGSLRLSCAASGFNFHRAYIHWVRQAPGKGLEWVASISSYSGYTYYADSVKGRFTISADTSKNTAYLQMNSLRAEDTAVYYCARSYQGQEALDYWGQGTLVTVSS

SEQ ID NO: 68 - protein, 122 aa, Artificial Sequence
  Artificial Polypeptide
EISEVQLVESGGGLVQPGGSLRLSCAASGFNFHRIYIHWVRQAPGKGLEWVASISSYSGYTYYADSVKGRFTISADTSKNTAYLQMNSLRAEDTAVYYCARSYQGQEALDYWGQGTLVTVSS

SEQ ID NO: 69 - protein, 122 aa, Artificial Sequence
  Artificial Polypeptide
EISEVQLVESGGGLVQPGGSLRLSCAASGFNFWASYIHWVRQAPGKGLEWVASISSYSGYTYYADSVKGRFTISADTSKNTAYLQMNSLRAEDTAVYYCARSYQGQEALDYWGQGTLVTVSS

SEQ ID NO: 70 - protein, 122 aa, Artificial Sequence
  Artificial Polypeptide
EISEVQLVESGGGLVQPGGSLRLSCAASGFNFYASYIHWVRQAPGKGLEWVASISSYSGYTYYADSVKGRFTISADTSKNTAYLQMNSLRAEDTAVYYCARSYQGQEALDYWGQGTLVTVSS

SEQ ID NO: 71 - protein, 122 aa, Artificial Sequence
  Artificial Polypeptide
EISEVQLVESGGGLVQPGGSLRLSCAASGFNLANNYVHWVRQAPGKGLEWVASISSYSGYTYYADSVKGRFTISADTSKNTAYLQMNSLRAEDTAVYYCARSYQGQEALDYWGQGTLVTVSS

SEQ ID NO: 72 - protein, 122 aa, Artificial Sequence
  Artificial Polypeptide
EISEVQLVESGGGLVQPGGSLRLSCAASGFNFHNSYIHWVRQAPGKGLEWVASISSYSGYTYYADSVKGRFTISADTSKNTAYLQMNSLRAEDTAVYYCARSYQGQEALDYWGQGTLVTVSS

SEQ ID NO: 73 - protein, 122 aa, Artificial Sequence
  Artificial Polypeptide
EISEVQLVESGGGLVQPGGSLRLSCAASGFNFHRTYIHWVRQAPGKGLEWVASISSYSGYTYYADSVKGRFTISADTSKNTAYLQMNSLRAEDTAVYYCARSYQGQEALDYWGQGTLVTVSS

SEQ ID NO: 74 - protein, 122 aa, Artificial Sequence
  Artificial Polypeptide
EISEVQLVESGGGLVQPGGSLRLSCAASGFNLNNIYIHWVRQAPGKGLEWVASISSYSGYTYYADSVKGRFTISADTSKNTAYLQMNSLRAEDTAVYYCARSYQGQEALDYWGQGTLVTVSS

SEQ ID NO: 75 - protein, 122 aa, Artificial Sequence
  Artificial Polypeptide
EISEVQLVESGGGLVQPGGSLRLSCAASGFNVHWVYFHWVRQAPGKGLEWVASISSYSGYTYYADSVKGRFTISADTSKNTAYLQMNSLRAEDTAVYYCARSYQGQEALDYWGQGTLVTVSS

SEQ ID NO: 76 - protein, 122 aa, Artificial Sequence
  Artificial Polypeptide
EISEVQLVESGGGLVQPGGSLRLSCAASGFNLHNQYLHWVRQAPGKGLEWVASISSYSGYTYYADSVKGRFTISADTSKNTAYLQMNSLRAEDTAVYYCARSYQGQEALDYWGQGTLVTVSS

SEQ ID NO: 77 - protein, 216 aa, Artificial Sequence
  Artificial Polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQYLYFSLVTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 216 aa, Artificial Sequence
  Artificial Polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQSNYSYPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 216 aa, Artificial Sequence
  Artificial Polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQASWGSPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 216 aa, Artificial Sequence
  Artificial Polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQSYYGSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 216 aa, Artificial Sequence
  Artificial Polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQSYYGNLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 216 aa, Artificial Sequence
  Artificial Polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQHAWGYLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 216 aa, Artificial Sequence
  Artificial Polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQYYYGALITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 230 aa, Artificial Sequence
  Artificial Polypeptide
EISEVQLVESGGGLVQPGGSLRLSCAASGFNFSSSYIHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 85 - protein, 228 aa, Artificial Sequence
  Artificial Polypeptide
EISEVQLVESGGGLVQPGGSLRLSCAASGFNVYSSYIHWVRQAPGKGLEWVAYISSYYGSTYYADSVKGRFTISADTSKNTAYLQMNSLRAEDTAVYYCARYAGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 86 - protein, 228 aa, Artificial Sequence
  Artificial Polypeptide
EISEVQLVESGGGLVQPGGSLRLSCAASGFNIYSSYIHWVRQAPGKGLEWVAYISSYYGSTYYADSVKGRFTISADTSKNTAYLQMNSLRAEDTAVYYCARYGDDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 87 - protein, 228 aa, Artificial Sequence
  Artificial Polypeptide
EISEVQLVESGGGLVQPGGSLRLSCAASGFNLYSSYIHWVRQAPGKGLEWVAYISSYYGSTYYADSVKGRFTISADTSKNTAYLQMNSLRAEDTAVYYCARYHSWAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 88 - protein, 229 aa, Artificial Sequence
  Artificial Polypeptide
EISEVQLVESGGGLVQPGGSLRLSCAASGFNVYSSYIHWVRQAPGKGLEWVAYISSYYGSTYYADSVKGRFTISADTSKNTAYLQMNSLRAEDTAVYYCARYSYPGGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 89 - protein, 229 aa, Artificial Sequence
  Artificial Polypeptide
EISEVQLVESGGGLVQPGGSLRLSCAASGFNVSSSYIHWVRQAPGKGLEWVAYISSYYGSTYYADSVKGRFTISADTSKNTAYLQMNSLRAEDTAVYYCARYAEGMGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 90 - protein, 229 aa, Artificial Sequence
  Artificial Polypeptide
EISEVQLVESGGGLVQPGGSLRLSCAASGFNVYSSYIHWVRQAPGKGLEWVAYISPYYGSTYYADSVKGRFTISADTSKNTAYLQMNSLRAEDTAVYYCARYWSYGGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 91 - protein, 230 aa, Artificial Sequence
  Artificial Polypeptide
EISEVQLVESGGGLVQPGGSLRLSCAASGFNFGGSYIHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 92 - protein, 230 aa, Artificial Sequence
  Artificial Polypeptide
EISEVQLVESGGGLVQPGGSLRLSCAASGFNFHGIYLHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 93 - protein, 230 aa, Artificial Sequence
  Artificial Polypeptide
EISEVQLVESGGGLVQPGGSLRLSCAASGFNFHSTYIHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 94 - protein, 230 aa, Artificial Sequence
  Artificial Polypeptide
EISEVQLVESGGGLVQPGGSLRLSCAASGFNFWLGYIHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 95 - protein, 230 aa, Artificial Sequence
  Artificial Polypeptide
EISEVQLVESGGGLVQPGGSLRLSCAASGFNFWNSYIHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 96 - protein, 230 aa, Artificial Sequence
  Artificial Polypeptide
EISEVQLVESGGGLVQPGGSLRLSCAASGFNFWTAYIHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 97 - protein, 230 aa, Artificial Sequence
  Artificial Polypeptide
EISEVQLVESGGGLVQPGGSLRLSCAASGFNFFNSYIHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 98 - protein, 230 aa, Artificial Sequence
  Artificial Polypeptide
EISEVQLVESGGGLVQPGGSLRLSCAASGFNFHNIYLHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 99 - protein, 230 aa, Artificial Sequence
  Artificial Polypeptide
EISEVQLVESGGGLVQPGGSLRLSCAASGFNFHNLYVHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 100 - protein, 230 aa, Artificial Sequence
  Artificial Polypeptide
EISEVQLVESGGGLVQPGGSLRLSCAASGFNFHRAYIHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 101 - protein, 230 aa, Artificial Sequence
  Artificial Polypeptide
EISEVQLVESGGGLVQPGGSLRLSCAASGFNFHRIYIHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 102 - protein, 230 aa, Artificial Sequence
  Artificial Polypeptide
EISEVQLVESGGGLVQPGGSLRLSCAASGFNFWASYIHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 103 - protein, 230 aa, Artificial Sequence
  Artificial Polypeptide
EISEVQLVESGGGLVQPGGSLRLSCAASGFNFYASYIHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 104 - protein, 230 aa, Artificial Sequence
  Artificial Polypeptide
EISEVQLVESGGGLVQPGGSLRLSCAASGFNLANNYVHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 105 - protein, 230 aa, Artificial Sequence
  Artificial Polypeptide
EISEVQLVESGGGLVQPGGSLRLSCAASGFNFHNSYIHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 106 - protein, 230 aa, Artificial Sequence
  Artificial Polypeptide
EISEVQLVESGGGLVQPGGSLRLSCAASGFNFHRTYIHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 107 - protein, 230 aa, Artificial Sequence
  Artificial Polypeptide
EISEVQLVESGGGLVQPGGSLRLSCAASGFNLNNIYIHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 108 - protein, 230 aa, Artificial Sequence
  Artificial Polypeptide
EISEVQLVESGGGLVQPGGSLRLSCAASGFNVHWVYFHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 109 - protein, 230 aa, Artificial Sequence
  Artificial Polypeptide
EISEVQLVESGGGLVQPGGSLRLSCAASGFNLHNQYLHWVRQAPGKGLEWVASISSYSGYTYYADSVKGRFTISADTSKNTAYLQMNSLRAEDTAVYYCARSYQGQEALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 110 - DNA, 29903 bp, Severe acute respiratory syndrome coronavirus 2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 111 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 112 - protein, 223 aa, Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 113 - protein, 10 aa, Artificial Sequence
  Artificial Polypeptide
YISSYYGSTY

SEQ ID NO: 114 - protein, 31 aa, Artificial Sequence
  Synthetic polypeptide
EISEVQLVESGGGLVQPGGSLRLSCAASGFN

SEQ ID NO: 115 - protein, 15 aa, Artificial Sequence
  Synthetic polypeptide
HWVRQAPGKGLEWVA

SEQ ID NO: 116 - protein, 39 aa, Artificial Sequence
  Synthetic polypeptide
YADSVKGRFTISADTSKNTAYLQMNSLRAEDTAVYYCAR

SEQ ID NO: 117 - protein, 13 aa, Artificial Sequence
  Synthetic polypeptide
DYWGQGTLVTVSS

SEQ ID NO: 118 - protein, 28 aa, Artificial Sequence
  Synthetic polypeptide
SDIQMTQSPSSLSASVGDRVTITCRASQ

SEQ ID NO: 119 - protein, 17 aa, Artificial Sequence
  Synthetic polypeptide
VAWYQQKPGKAPKLLIY

SEQ ID NO: 120 - protein, 34 aa, Artificial Sequence
  Synthetic polypeptide
GVPSRFSGSRSGTDFTLTISSLQPEDFATYYCQQ

SEQ ID NO: 121 - protein, 11 aa, Artificial Sequence
  Synthetic polypeptide
TFGQGTKVEIK